(19) United States
(12) Patent Application Publication
(10) Pub. No.: US 2008/0305082 A1
(43) Pub. Date: Dec. 11, 2008

(54) 1,4-Bis-N-Oxide-5,8- Dihydroxyanthracenedione Compounds and the Use Thereof

(76) Inventors: John G. Curd, Hillsborough, CA (US); Robert L. Capizzi, Philadelphia, PA (US); John F.W. Keana, Eugene, OR (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/662,599

(22) PCT Filed: Sep. 14, 2005
(86) PCT No.: PCT/US05/32398

§ 371 Date: Mar. 13, 2008

Related U.S. Application Data
(60) Provisional application No. 60/609,253, filed on Sep. 14, 2004.

(51) Int. Cl. A61K 38/21; C07C 211/44; C07D 205/04; C07D 207/04; C07D 295/00; C07D 243/08; C07D 263/04; C07D 241/36; C07D 221/02; C07D 279/14; A61K 31/136; A61K 31/397; A61K 31/4025; A61K 31/496; A61K 31/551; A61K 31/40; A61K 31/421; A61K 31/498; A61P 35/00; A61K 31/435; A61K 31/542; A61K 31/592

(52) U.S. Cl. 424/85.7; 564/427; 548/950; 548/518; 544/357; 540/575; 548/558; 548/233; 544/343; 546/78; 544/14; 544/342; 546/49; 514/656; 514/210.2; 514/422; 514/252.11; 514/218; 514/424; 514/377; 514/250; 514/284; 514/224.5; 514/280; 514/167

(57) ABSTRACT

Disclosed are compounds having Formula I: and pharmaceutically acceptable salts thereof, wherein R₁, R₂, R₃, R₄, R₅, R₆, R₇, R₈, R₉, R₁₀, A, and B are as defined herein, for use in methods for treating, preventing or ameliorating hyperproliferative disorders, such as cancer and other diseases and conditions. The invention also relates to pharmaceutical compositions and formulations comprising a compound having Formula I, and in combination with one or more other active agents and/or treatments.

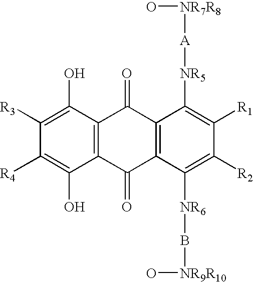

### BACKGROUND OF THE INVENTION

1. Field of the Invention

The present invention relates to novel compounds having activity for treating hyperproliferative disorders, including neoplastic and non-neoplastic disorders, as well as certain inflammatory conditions. The invention also relates to pharmaceutical compositions and formulations comprising the novel compounds. Further, the invention relates to methods of using the novel compounds, alone or in combination with one or more other active agents or treatments, to treat hyperproliferative disorders, including various cancers.

2. Related Art

One in every four deaths in the United States is due to cancer, and cancer is the second leading cause of death. U.S. Cancer Statistics Working Group; United States Cancer Statistics: 2000 Incidence, Atlanta (GA): Department of Health and Human Services, Centers for Disease Control and Prevention, and National Cancer Institute (2003). The National Cancer Institute reports that almost 10 million Americans have a history of invasive cancer, while the American Cancer Society estimates that in the year 2004, over 1.3 million Americans will receive a diagnosis of invasive cancer with over a half million cases resulting in death. American Cancer Society, Cancer Facts & Figures 2004. These statistics exclude the 1 million cases of basal and squamous cell skin cancers that are expected to be diagnosed in the United States.

Cancers are classified based on the organ and cell tissue from which the cancer originates, including: (i) carcinomas (most common kind of cancer which originates in epithelial tissues, the layers of cells covering the body's surface or lining internal organs and various glands); (ii) leukemias (origination in the blood-forming tissues, including bone marrow, lymph nodes and the spleen); (iii) lymphomas (originates in the cells of the lymph system); (iv) melanomas (originates in the pigment cells located among the epithelial cells of the skin); and (v) sarcomas (originates in the connective tissues of the body, such as bones, muscles and blood vessels). (See Molecular Biology of the Cell: Third Edition, “Cancer,” Chapter 24, pp. 1255-1294, B. Alberts et al., (eds.), Garland Publishing, Inc., New York (1994); and Stedman's Pocket Medical Dictionary; Williams and Wilkins, Baltimore (1987)). Within these broad cancer classifications, there are over one hundred cancer subclassifications, such as breast, lung, pancreatic, colon, and prostate cancer, to name a few.

Cancer cells develop as a result of damage to a cell's DNA (i.e., altered DNA sequence or altered expression pattern) from exposure to various chemical agents, radiation, viruses, or when some not-yet-fully-understood internal, cellular signaling event occurs. Most of the time when a cell's DNA becomes damaged, the cell either dies or is able to repair the DNA. However, for cancer cells, the damaged DNA is not repaired and the cell continues to divide, exhibiting modified cell physiology and function.

Neoplasms, or tumors, are masses of cells that result from an aberrant, accelerated rate of growth (i.e., hyperproliferative cell growth). As long as the tumor cells remain confined to a single mass, the tumor is considered to be benign. However, a cancerous tumor has the ability to invade other tissues and is termed malignant. In general, cancer cells are defined by two heritable properties: the cells and their progeny 1) reproduce in defiance of normal restraints, and 2) invade and colonize the territories of other cells.

Cancerous tumors are comprised of a highly complex vasculature and differentiated tissue. A large majority of cancerous tumors have hypoxic components, which are relatively resistant to standard anti-cancer treatment, including radiotherapy and chemotherapy. Brown, Cancer Res. 59:5863 (1999); and Kunz, M. et al., Mol. Cancer. 2:1 (2003). Thomlinson and Gray presented the first anatomical model of a human tumor that describes a 100 to 150 μm thick hypoxic layer of tissue located between the blood vessels and necrotic tumor tissues.

Research has shown that the hypoxic tissues within a number of cancerous tumors promote the progression of the cancer by an array of complex mechanisms. See, Brown., supra, and Kunz et al., supra. Among these are activation of certain signal transduction pathways and gene regulatory mechanisms, induction of selection processes for gene mutations, tumor cell apoptosis and tumor angiogenesis. Most of these mechanisms contribute to tumor progression. Therefore, tissue hypoxia has been regarded as a central factor for tumor aggressiveness and metastasis. Therapies that target hypoxic tissues within a tumor would certainly provide improved treatments to patients suffering from tumor-related cancers and/or disorders.

In addition to cancer, there exist a number of hyperproliferative diseases and/or disorders that are associated with the onset of hypoxia in a given tissue. For example, Shweiki et al. explains that inadequate oxygen levels often lead to neovascularization in order to compensate for the needs of the hypoxic tissue. Neovascularization is mediated by expression of certain growth factors, such as vascular endothelial growth factor (VEGF). Shweiki et al., Nature 359:843 (1992). However, when certain tissues or growth factors are either directly or indirectly upregulated in response to hypoxia without sufficient feedback mechanisms for controlling tissue expression, various diseases and/or disorders may ensue (i.e., by hypoxia-aggravated hyperproliferation). By way of example, hypoxia-aggravated hyperproliferative diseases and/or disorders having over-expressed levels of VEGF include ocular angiogenic diseases, such as age-related macular degeneration and diabetic retinopathy, as well as cirrhosis of the liver. See Frank, Ophthalmic Res. 29:341 (1997); Ishibashi et al., Graefe's Archive Clin. Exp. Ophthamol. 235:159 (1997); Corpechot et al., Hepatology 35:1010 (2002).

U.S. Pat. No. 5,132,327 describes a group of anthraquinone prodrug compounds having the following structure:

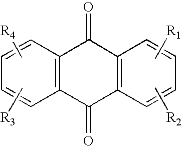

in which R₁, R₂, R₃ and R₄ are each separately selected from the group consisting of hydrogen, X, NH-A-NHR and NH-A-N(O)R′R″ wherein X is hydroxy, halogeno, amino, C₁₋₄ alkoxy or C₂₋₈ alkanoyloxy, A is a C₂₋₄ alkylene group with a chain length between NH and NHR or N(O)R′R″ of at least 2 carbon atoms and R, R′ and R″ are each separately selected from the group consisting of C₁₋₄ alkyl groups and C₂₋₄ hydroxyalkyl and C₂₋₄ dihydroxyalkyl groups in which the carbon atom attached to the nitrogen atom does not carry a hydroxy group and no carbon atom is substituted by two hydroxy groups, or R′ and R″ together are a C₂₋₆ alkylene group which with the nitrogen atom to which R′ and R″ are attached forms a heterocyclic group having 3 to 7 atoms in the ring, but with the proviso that at least one of R₁ to R₄ is a group NH-A-N(O)R′R″, the compound optionally being in the form of a physiologically acceptable salt. These compounds are described as being useful in the treatment of cancer.

Among the compounds disclosed in U.S. Pat. No. 5,132,327 is the compound AQ4N (1,4-bis{[2-(dimethylamino)ethyl]amino}-5,8-dihydroxyanthracene-9,10-dione bis-N-oxide) (compound 1).

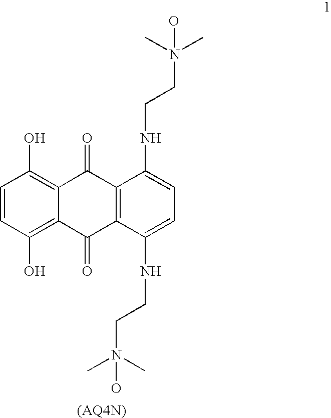

AQ4N has been shown to have potent anti-hyperproliferative activity and to enhance the antitumor effects of radiation and conventional chemotherapeutic agents. Patterson, Drug Metab. Rev. 34:581 (2002). For many tumor cells, AQ4N is not intrinsically cytotoxic; in hypoxic tumors it is converted to the cytotoxic compound AQ4 (1,4-bis{[2-(dimethylamino)ethyl]amino}-5,8-dihydroxyanthracene-9,10-dione). Among the activities associated with AQ4 are intercalation into DNA and inhibition of topoisomerase II activity.

### BRIEF SUMMARY OF THE INVENTION

The present invention is related to compounds, compositions and methods for treating hyperproliferative disorders, such as cancer and inflammation. One aspect of the invention is drawn to compounds having Formula I:

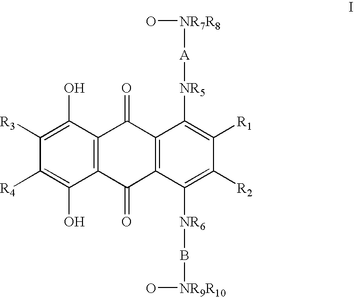

or a pharmaceutically acceptable salt thereof, wherein:

R₁ and R₂ are independently hydrogen, halo, alkyl, carboxyl, carboxylester, carboxylamide, thioalkyl, hydroxy, alkoxy, aryloxy, sulfonyl, sulfonic acid, or polyethylene glycol, or R₁ and R₂ together form an aryl or heteroaryl group;

R₃ and R₄ independently are hydrogen or fluorine, or R₃ and R₄ together form an aryl group;

R₅ and R₆ independently are hydrogen, alkyl, or hydroxyalkyl;

or R₁ and R₅ together, and/or R₂ and R₆ together, form a ring;

R₇ and R₈ are independently alkyl, hydroxyalkyl, haloalkyl, or together with the neighboring nitrogen form a heterocycle;

R₉ and R₁₀ are independently alkyl, hydroxyalkyl, haloalkyl, or together with the neighboring nitrogen form a heterocycle;

A and B independently are (CH₂)ₙ, cycloalkyl, heterocyclic, or aryl, or with the two neighboring nitrogens forms a heterocycle; and

n is 1, 2, 3, or 4;

with the proviso that at least one of R₁, R₂, R₃, R₄, R₅ and R₆ is other than hydrogen; or

at least one of R₇, R₈, R₉, and R₁₀ is haloalkyl;

or R₇ and R₈, and/or R₉ and R₁₀, form a heterocycle with the neighboring nitrogen; or

at least one of A and B is cycloalkyl, heterocyclic or aryl or forms a heterocycle with the two neighboring nitrogens.

According to another aspect of the invention, a therapeutically effective amount of a compound having Formula I is provided in the form of a pharmaceutical composition having at least one pharmaceutically acceptable carrier.

In another aspect of the invention, methods for treating, ameliorating, or preventing hyperproliferative disorders are provided, wherein a therapeutically effective amount of a compound having Formula I, or a pharmaceutically acceptable salt thereof, is administered to an animal in need thereof. Additionally, the invention can be practiced by formulation of a compound having Formula I and at least one other active agent optionally as part of a single pharmaceutical composition.

An additional aspect of the present invention is a method for treating, ameliorating, or preventing hyperproliferative disorders in an animal comprising administering to the animal a therapeutically effective amount of a compound having Formula I in combination with one or more active agents or treatments, for example, chemotherapeutic agents or radiotherapeutic agents/treatments.

In preferred embodiments of the invention, the one or more chemotherapeutic agents can be any chemotherapeutic agent which is used, has been used, or is known to be useful for the treatment of hyperproliferative disorders.

In preferred embodiments of the invention, the one or more radiotherapeutic agents or treatments can be external-beam radiation therapy, brachytherapy, thermotherapy, radiosurgery, charged-particle radiotherapy, neutron radiotherapy, photodynamic therapy, or radionuclide therapy.

In one embodiment of the invention, the compound having Formula I can be administered prior to, during, and/or beyond administration of the one or more chemotherapeutic agents or radiotherapeutic agents or treatments. In another embodiment of the invention, the method of administering a compound having Formula I in combination with one or more chemotherapeutic agents or radiotherapeutic agents or treatments is repeated more than once.

The combination of a compound having Formula I and one or more chemotherapeutic agents or radiotherapeutic agents or treatments of the present invention will have additive potency or an additive therapeutic effect. The invention also encompasses synergistic combinations where the therapeutic efficacy is greater than additive. Preferably, such combinations will reduce or avoid unwanted or adverse effects. In certain embodiments, the combination therapies encompassed by the invention will provide an improved overall therapy relative to administration of a compound having Formula I or any chemotherapeutic agent or radiotherapeutic agent or treatment alone. In certain embodiments, doses of existing or experimental chemotherapeutic agents or radiotherapeutic agents or treatments will be reduced or administered less frequently which will increase patient compliance, thereby improving therapy and reducing unwanted or adverse effects.

Further, the methods of the invention will be useful not only with previously untreated patients but also will be useful in the treatment of patients partially or completely refractory to current standard and/or experimental cancer therapies, including but not limited to radiotherapies, chemotherapies, and/or surgery. In a preferred embodiment, the invention will provide therapeutic methods for the treatment or amelioration of hyperproliferative disorders that have been shown to be or may be refractory or non-responsive to other therapies.

While not wishing to be bound by any theory, it is believed that some of the N-oxide compounds of the invention will function as prodrugs with greatly diminished cytotoxicity. It is believed that these N-oxide compounds will be activated under hypoxic conditions within the target tissues (i.e., reduced at the nitrogen atom), followed by intercalation between the base pairs in the host cell DNA. Other N-oxide compound of the invention may have intrinsic cytotoxic activity. It is contemplated that the targets of the compounds for facilitating cell toxicity include DNA, helicases, microtubules, protein kinase C, and topoisomerase I and II. Since a number of pathological tissues have significant hypoxic components which promote hyperproliferation, it is believed that this portion of tissue will be preferentially targeted. It is also believed that compounds having Formula I will be useful for the treatment, prevention or amelioration of any number of hypoxia-aggravated hyperproliferative diseases and/or disorders. Such diseases and/or disorders include, without limitation, age-related macular degeneration, various cancers, Crohn's disease, cirrhosis, chronic inflammatory-related disorders, diabetic retinopathy, granulomatosis, immune hyperproliferation associated with organ or tissue transplantation, inflammatory bowel disease, psoriasis, rheumatoid arthritis, systemic lupus erythematosus, vascular hyperproliferation secondary to retinal hypoxia, vascular hyperproliferation secondary to retinal hypoxia, and vasculitis.

### DETAILED DESCRIPTION OF THE INVENTION

One aspect of the invention is drawn to compounds having Formula I:

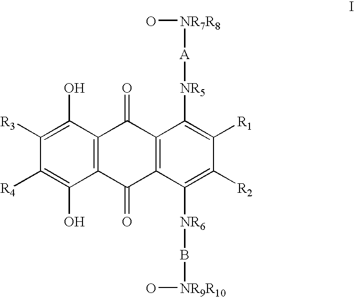

or a pharmaceutically acceptable salt thereof, wherein:

R₁ and R₂ are independently hydrogen, halo, alkyl, carboxyl, carboxylester, carboxylamide, thioalkyl, hydroxy, alkoxy, aryloxy, sulfonyl, sulfonic acid, or polyethylene glycol, or R₁ and R₂ together form an aryl or heteroaryl group;

R₃ and R₄ independently are hydrogen or fluorine, or R₃ and R₄ together form an aryl group;

R₅ and R₆ independently are hydrogen, alkyl, or hydroxyalkyl;

or R₁ and R₅ together, and/or R₂ and R₆ together, form a ring;

R₇ and R₉ are independently alkyl, hydroxyalkyl, haloalkyl, or together with the neighboring nitrogen form a heterocycle;

R₉ and R₁₀ are independently alkyl, hydroxyalkyl, haloalkyl, or together with the neighboring nitrogen form a heterocycle;

A and B independently are (CH₂)ₙ, cycloalkyl, or aryl, or with the two neighboring nitrogens forms a heterocycle; and

n is 1, 2, 3, or 4;

with the proviso that at least one of R₁, R₂, R₃, R₄, R₅ and R₆ is other than hydrogen; or

at least one of R₇, R₈, R₉, and R₁₀ is haloalkyl;

or R₇ and R₈, and/or R₉ and R₁₀, form a heterocycle with the neighboring nitrogen; or

at least one of A and B is cycloalkyl, heterocyclic or aryl or forms a heterocycle with the two neighboring nitrogens.

In one embodiment the invention is drawn to compounds having Formula II:

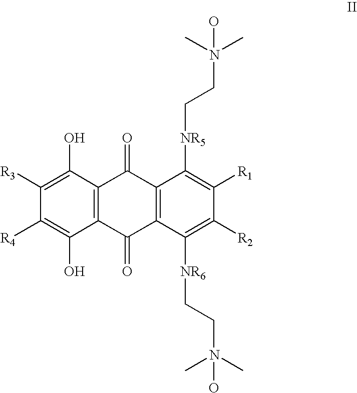

wherein R₁ is hydrogen, halo or optionally substituted alkyl or alkoxy, and R₂, R₃, and R₄ are hydrogen or halo. In particular embodiments the compounds are selected from the compounds shown below.

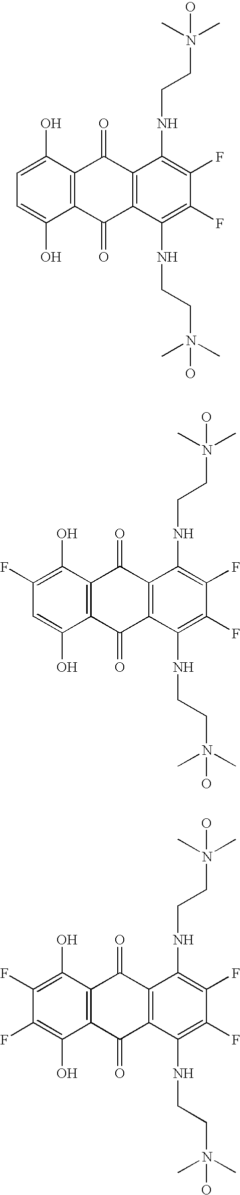

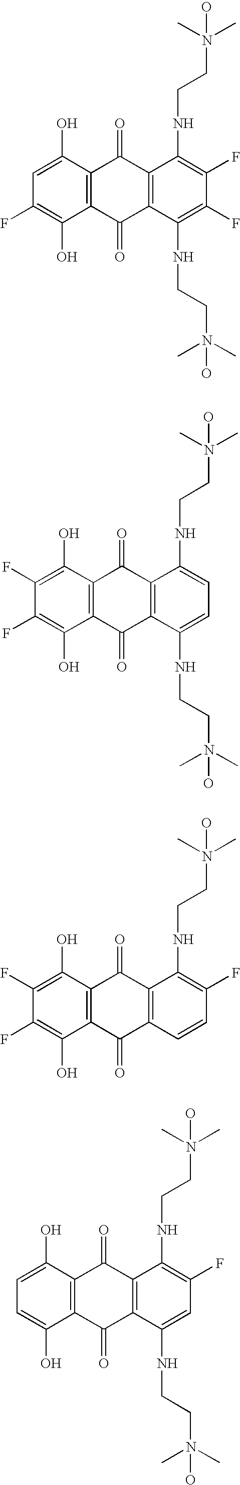

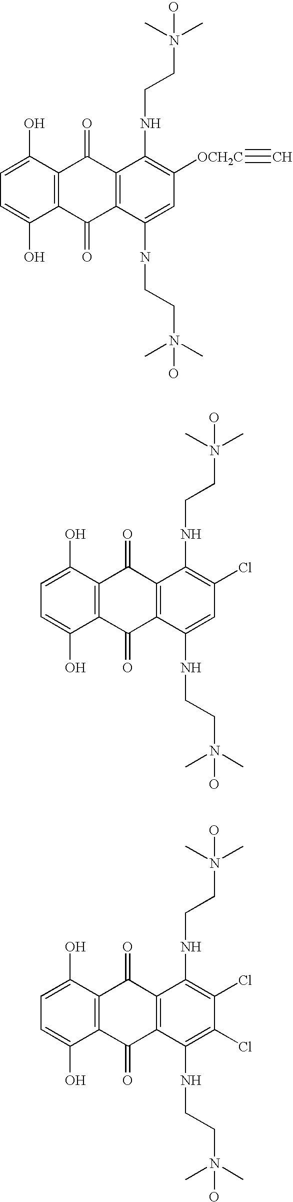

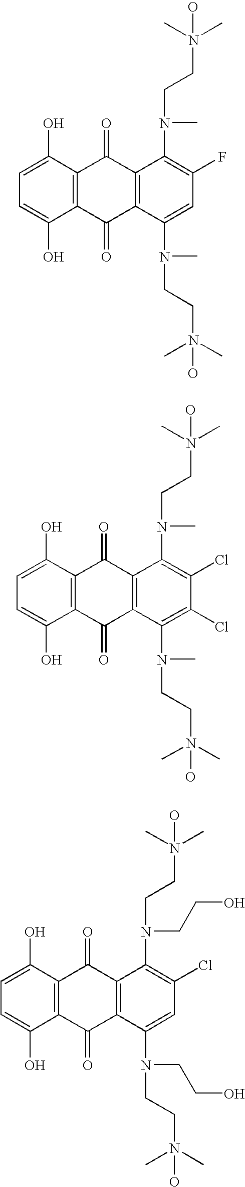

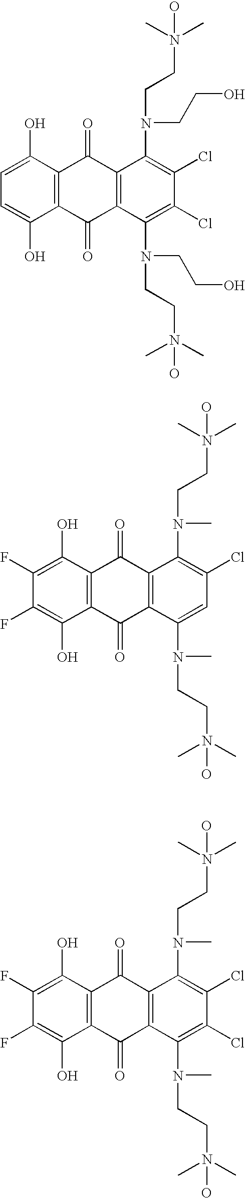

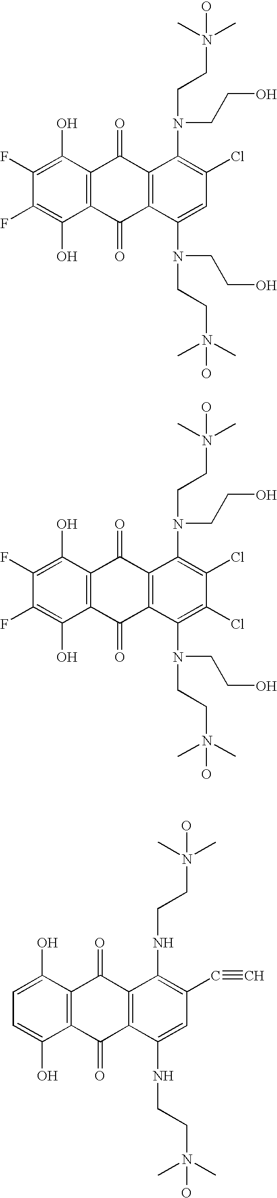

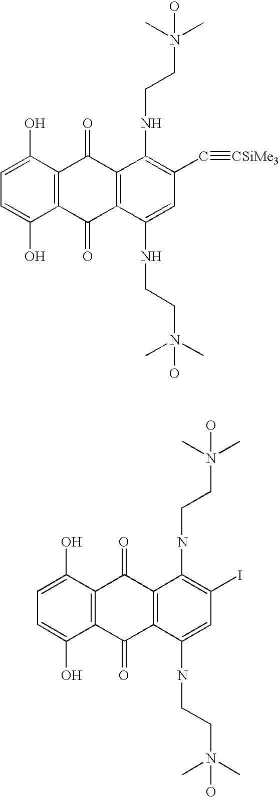

In another aspect the invention is drawn to compounds having Formula III:

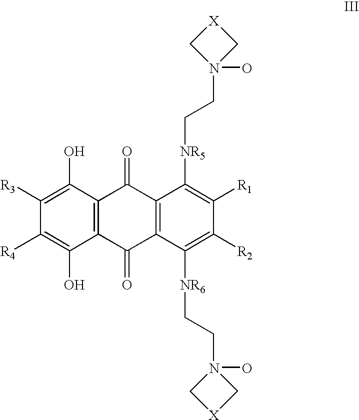

wherein X is (CH₂)ₘ and m is 0 to 5. In particular embodiments the compounds are selected from the compounds shown below.

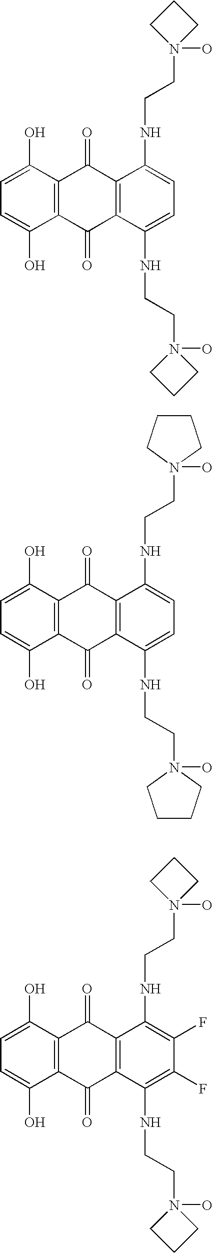

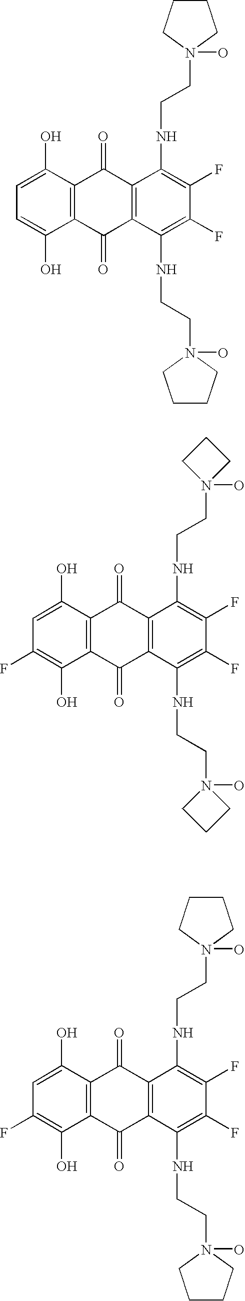

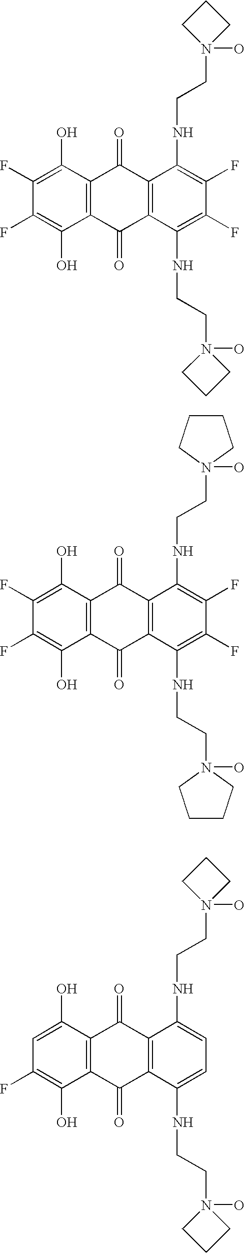

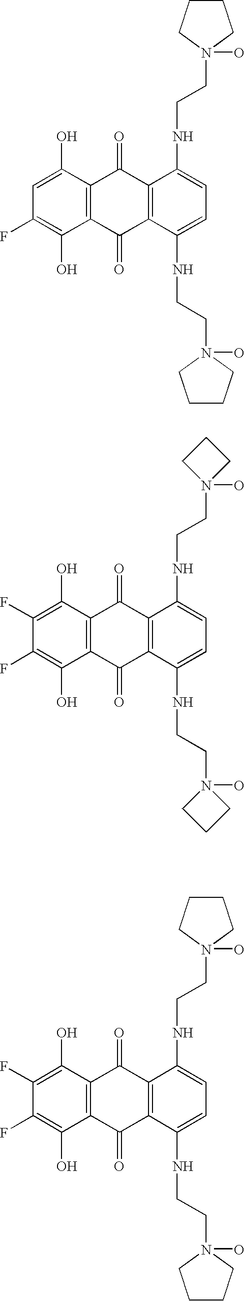

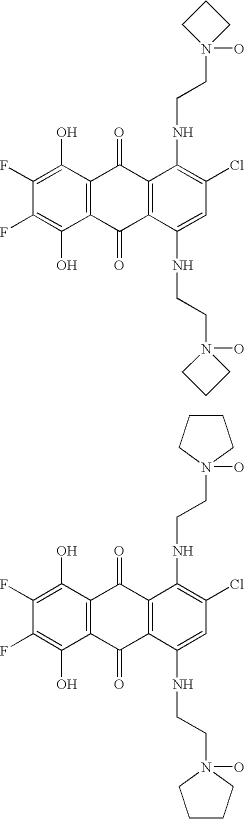

In another aspect the invention is drawn to compounds having Formula IV:

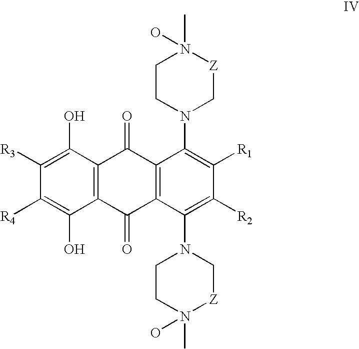

wherein Z is (CH₂)ₘ and m is 0 to 5. In particular embodiments the compounds are selected from the compounds shown below.

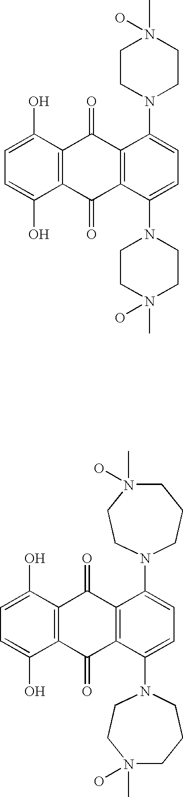

In another embodiment the invention is drawn to compounds having Formula V:

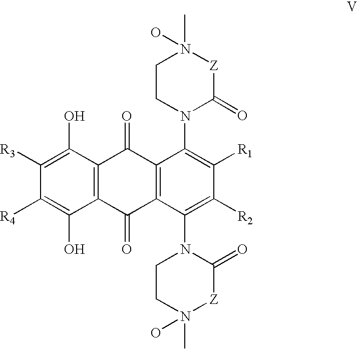

wherein Z is (CH₂)ₘ and m is 0 to 5. In particular embodiments the compounds are selected from the compounds shown below.

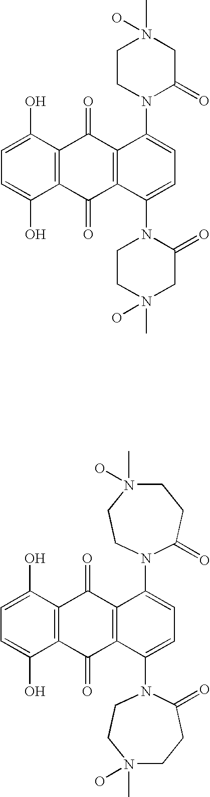

In another aspect the invention is drawn to compounds having Formula VI:

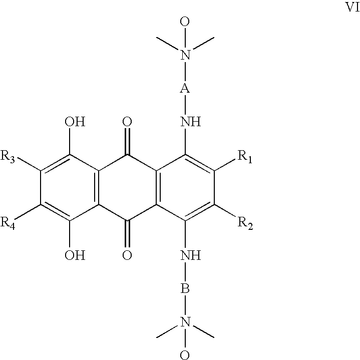

wherein A and B are independently cycloalkyl or heterocyclic groups containing 4-12 ring atoms. In particular embodiments the compounds are selected from the compounds shown below.

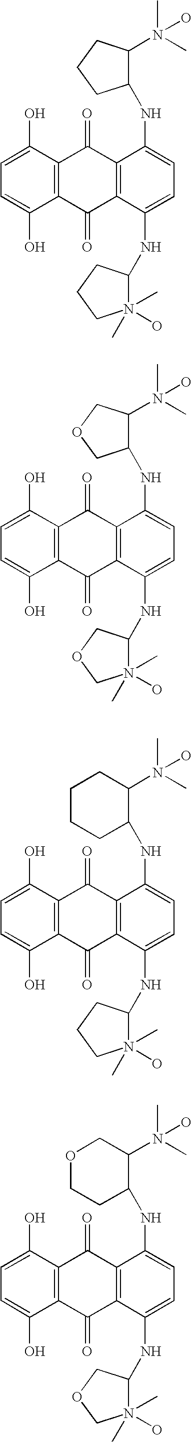

In another aspect the invention is drawn to compounds having Formula VII:

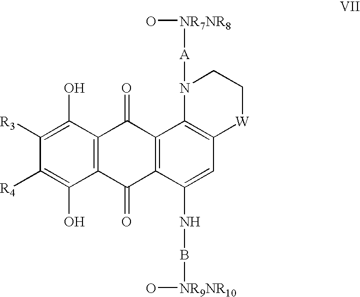

wherein W is CH₂, O, S, or NR₁₁, wherein R₁₁ is hydrogen or alkyl. In particular embodiments the compounds are selected from the compounds shown below.

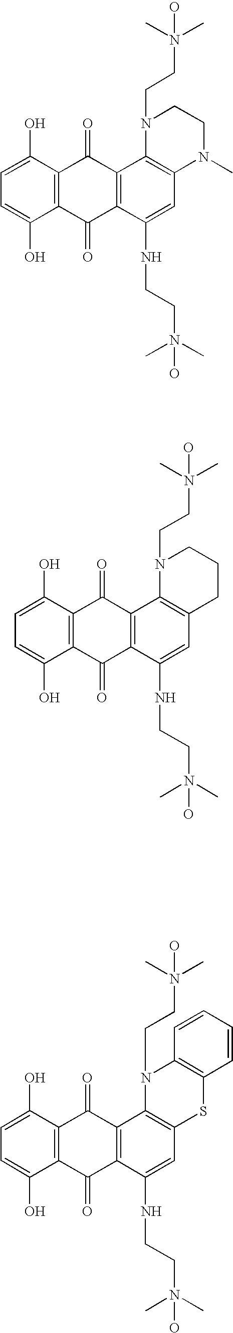

In another aspect the invention is drawn to compounds having Formula VIII:

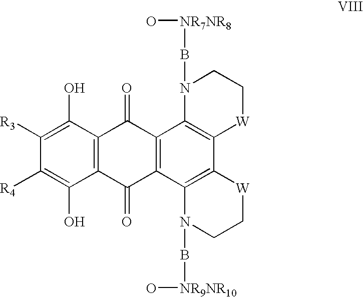

wherein W is CH₂, O, S, or NR₁₁, wherein R₁₁ is hydrogen or alkyl. In particular embodiments the compounds are selected from the compounds shown below.

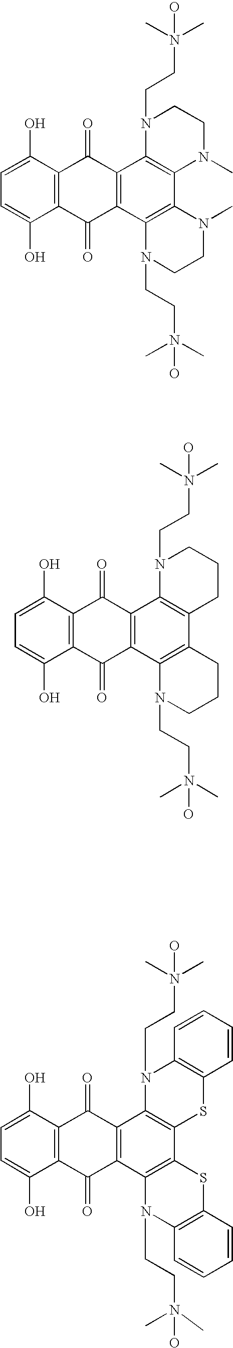

In another embodiment the invention is drawn to compounds having Formula IX:

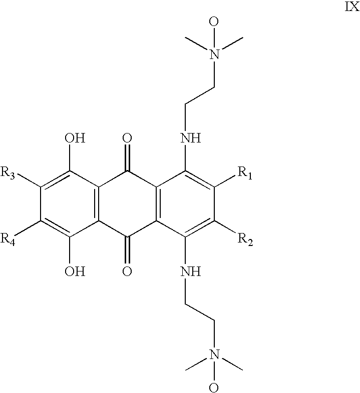

wherein R₁ and R₂ together and/or R₃ and R₄ together form an aryl or heteroaryl group. In particular embodiments the compounds are selected from the compounds shown below.

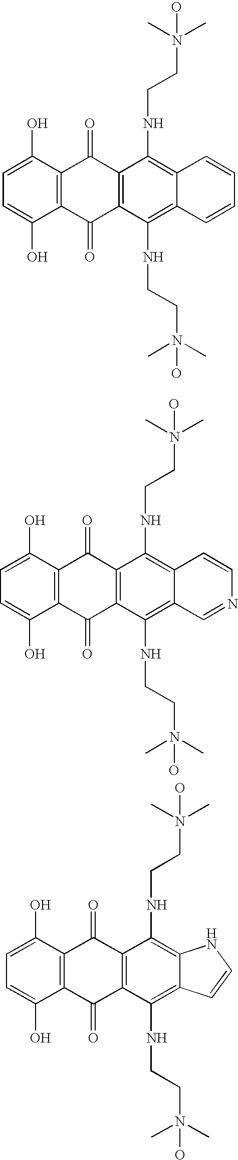

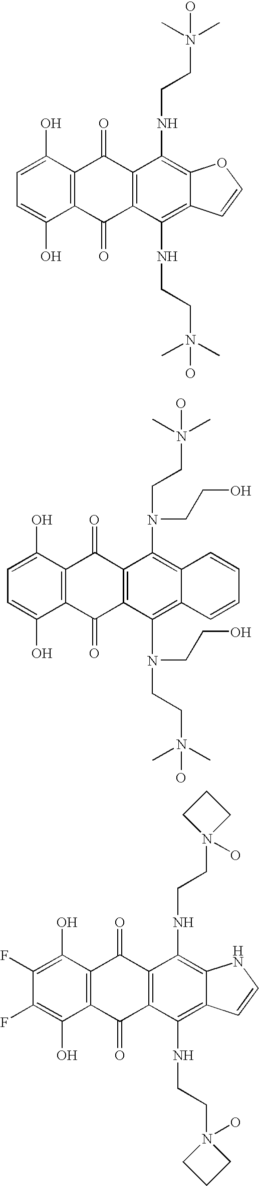

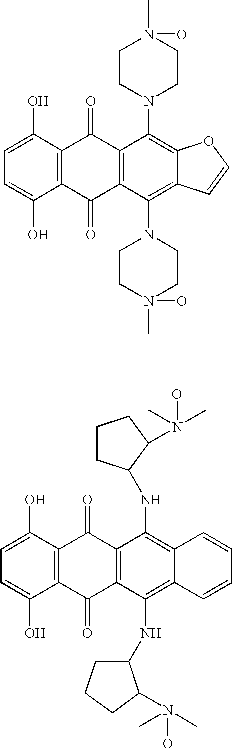

In another aspect the invention is drawn to compounds having Formula X:

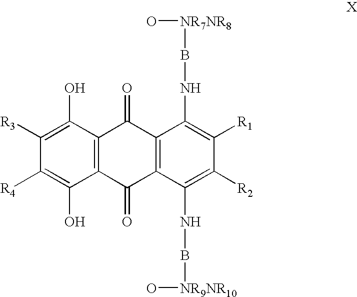

wherein at least one of R₇, R₈, R₉, and R₁₀ is haloalkyl. In particular embodiments the compounds are selected from the compounds shown below.

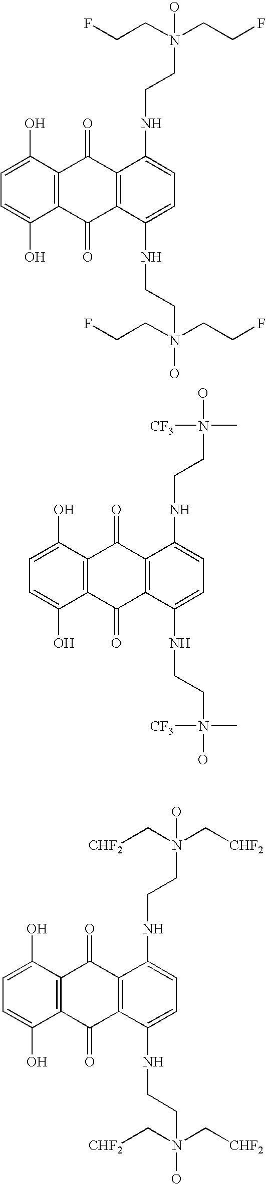

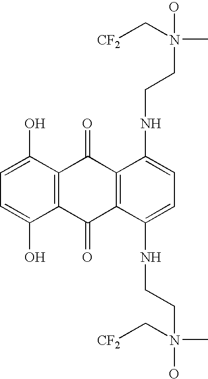

Useful alkyl groups include straight-chained or branched C₁₋₁₀ alkyl groups, especially methyl, ethyl, propyl, isopropyl, t-butyl, sec-butyl, 3-pentyl, adamantyl, norbornyl, and 3-hexyl groups.

Useful aryl groups include C₆₋₁₄ aryl, especially phenyl, naphthyl, phenanthrenyl, anthracenyl, indenyl, azulenyl, biphenyl, biphenylenyl, and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful halo or halogen groups include fluorine, chlorine, bromine and iodine.

Useful haloalkyl groups include C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful alkoxy groups include oxygen substituted by one of the C₁₋₁₀ alkyl groups mentioned above, which may be optionally substituted.

Useful alkylthio groups include sulphur substituted by one of the C₁₋₁₀ alkyl groups mentioned above, which may be optionally substituted. Also included are the sulfoxides and sulfones of such alkylthio groups.

Useful heterocyclic groups include tetrahydrofuranyl, pyranyl, piperidinyl, piperizinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

According to another aspect of the invention, a therapeutically effective amount of a compound having Formula I, or a pharmaceutically acceptable salt thereof, is provided in the form of a pharmaceutical composition having at least one pharmaceutically acceptable carrier.

In a further aspect of the invention, the pharmaceutical composition comprises a compound having Formula I and at least one other active agent. In certain instances, the at least one other active agent is a chemotherapeutic agent (including an active vitamin D compound) or anti-inflammatory agent. Compounds having Formula I may be formulated in a single formulation with the other active agent(s), or formulated independently.

According to one aspect of the invention, methods for treating, ameliorating, or preventing hyperproliferative disorders are provided, wherein a therapeutically effective amount of a compound having Formula I, or a pharmaceutically acceptable salt thereof, is administered to an animal in need thereof. In certain aspects of the invention, the hyperproliferative disorder is cancer. In certain other aspects of the invention, the hyperproliferative disorder is any one of age-related macular degeneration, Crohn's disease, cirrhosis, chronic inflammatory-related disorders, diabetic retinopathy, granulomatosis, immune hyperproliferation associated with organ or tissue transplantation, an immunoproliferative disease or disorder, e.g., inflammatory bowel disease, psoriasis, rheumatoid arthritis, systemic lupus erythematosus, vascular hyperproliferation secondary to retinal hypoxia, or vasculitis.

A further aspect of the invention relates to methods for treating, ameliorating, or preventing a hyperproliferative disorder comprising administering a therapeutically effective amount of a compound having Formula I, or a pharmaceutically acceptable salt thereof, in combination with at least one other active agent or treatment to a patient in need thereof.

Hyperproliferative disorders which can be treated with the compounds having Formula I include any hypoxia-aggravated hyperproliferative disease and/or disorder, such as any number of cancers. Generally, such cancers include, without limitation, cancers of the bladder, brain, breast, cervix, colon, endometrium, esophagus, head and neck, kidney, larynx, liver, lung, oral cavity, ovaries, pancreas, prostate, skin, stomach, and testis. Certain of these cancers may be more specifically referred to as acute and chronic lymphocytic leukemia, acute granulocytic leukemia, adrenal cortex carcinoma, bladder carcinoma, breast carcinoma, cervical carcinoma, cervical hyperplasia, choriocarcinoma, chronic granulocytic leukemia, chronic lymphocytic leukemia, colon carcinoma, endometrial carcinoma, esophageal carcinoma, essential thrombocytosis, genitourinary carcinoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, multiple myeloma, mycosis fungoides, myeloid and lymphocytic leukemia, neuroblastoma, non-Hodgkin's lymphoma, osteogenic sarcoma, ovarian carcinoma, pancreatic carcinoma, polycythemia vera, primary brain carcinoma, primary macroglobulinemia, prostatic carcinoma, renal cell carcinoma, rhabdomyosarcoma, skin cancer, small-cell lung carcinoma, soft-tissue sarcoma, squamous cell carcinoma, stomach carcinoma, testicular carcinoma, thyroid carcinoma, and Wilms' tumor.

Animals which may be treated according to the present invention include all animals which may benefit from administration of compounds having Formula I. Such animals include humans, pets such as dogs and cats, and veterinary animals such as cows, pigs, sheep, goats and the like.

The term “pharmaceutical composition” as used herein, is to be understood as defining compositions of which the individual components or ingredients are themselves pharmaceutically acceptable, e.g., where oral administration is foreseen, acceptable for oral use; where topical administration is foreseen, topically acceptable; and where intravenous administration is foreseen, intravenously acceptable.

As used herein, the term “therapeutically effective amount” refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

Compounds having Formula I can be provided as pharmaceutically acceptable salts. Examples of pharmaceutically acceptable salts (i.e., addition salts) include inorganic and organic acid addition salts such as hydrochloride, hydrobromide, phosphate, sulphate, citrate, lactate, tartrate, maleate, fumarate, mandelate, benzoate and oxalate; and inorganic and organic base addition salts with bases such as sodium hydroxy, Tris(hydroxymethyl)aminomethane (TRIS, tromethane) and N-methyl-glucamine. Although the salts typically have similar physiological properties compared to the free base, certain acid addition salts may demonstrate preferred physicochemical properties, e.g., enhanced solubility, improved stability. One particular pharmaceutically acceptable salt is the maleate, such as the dimaleate.

Certain of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

In certain embodiments of the invention, compounds having Formula I are administered in combination with one or more other active agents (e.g., chemotherapeutic or anti-inflammatory agents) or treatments. By way of non-limiting example, a patient may be treated for a hyperproliferative disorder, such as cancer, by the administration of a therapeutically effective amount of a compound having Formula I in combination with radiotherapy agent/treatment or the administration of a chemotherapeutic agent.

“In combination” refers to the use of more than one treatment. The use of the term “in combination” does not restrict the order in which treatments are administered to a subject being treated for a hyperproliferative disorder. A first treatment can be administered prior to, concurrently with, after, or within any cycling regimen involving the administration of a second treatment to a subject with a hyperproliferative disorder. For example, the first treatment can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before a treatment; or the first treatment can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after a second treatment. Such treatments include, for example, the administration of compounds having Formula I in combination with one or more chemotherapeutic agents or radiotherapeutic agents/treatments.

The term “chemotherapeutic agent,” as used herein, is intended to refer to any chemotherapeutic agent known to those of skill in the art to be effective for the treatment, prevention or amelioration of hyperproliferative disorders such as cancer. Chemotherapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. Any agent which is known to be useful, or which has been used or is currently being used for the treatment or amelioration of a hyperproliferative disorder can be used in combination with a compound having Formula I. See, e.g., Hardman et al., eds., 1996, Goodman & Gilman's The Pharmacological Basis Of Therapeutics 9th Ed, Mc-Graw-Hill, New York, N.Y. for information regarding therapeutic agents which have been or are currently being used for the treatment or amelioration of a hyperproliferative disorder.

Particular chemotherapeutic agents useful in the methods and compositions of the invention include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, angiogenesis inhibitors and anti-angiogenic agents.

Certain chemotherapeutic agents include, but are not limited to, abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

Chemotherapeutic agents may be administered at doses that are recognized by those of skill in the art to be effective for the treatment of the hyperproliferative disorder. In certain embodiments, chemotherapeutic agents may be administered at doses lower than those used in the art due to the additive or synergistic effect of the compounds having Formula I.

The term “active vitamin D compound,” as used herein, is intended to refer to vitamin D which has been hydroxylated in at least the carbon-1 position of the A ring, e.g., 1α-hydroxyvitamin D₃. One particular active vitamin D compound for use in the present invention is 1α,25-dihydroxyvitamin D₃, also known as calcitriol. A large number of other active vitamin D compounds are known and can be used in the practice of the invention. Examples include 1α-hydroxy derivatives with a 17 side chain greater in length than the cholesterol or ergosterol side chains (see U.S. Pat. No. 4,717,721); cyclopentano-vitamin D analogs (see U.S. Pat. No. 4,851,401); vitamin D₃ analogues with alkynyl, alkenyl, and alkanyl side chains (see U.S. Pat. Nos. 4,866,048 and 5,145,846); trihydroxycalciferol (see U.S. Pat. No. 5,120,722); fluoro-cholecalciferol compounds (see U.S. Pat. No. 5,547,947); methyl substituted vitamin D (see U.S. Pat. No. 5,446,035); 23-oxa-derivatives (see U.S. Pat. No. 5,411,949); 19-nor-vitamin D compounds (see U.S. Pat. No. 5,237,110); and hydroxylated 24-homo-vitamin D derivatives (see U.S. Pat. No. 4,857,518). Particular examples include ROCALTROL (Roche Laboratories); CALCIJEX injectable calcitriol; investigational drugs from Leo Pharmaceuticals including EB 1089 (24a,26a,27a-trihomo-22,24-diene-1α,25-(OH)₂-D₃, KH 1060 (20-epi-22-oxa-24a,26a,27a-trihomo-1α,25-(OH)₂-D₃), Seocalcitol, MC 1288 (1,25-(OH)₂-20-epi-D₃) and MC 903 (calcipotriol, 1α,24s-(OH)₂-22-ene-26,27-dehydro-D₃); Roche Pharmaceutical drugs that include 1,25-(OH)₂-16-ene-D₃, 1,25-(OH)₂-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃ and 1α-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25-(OH)₃-D₃; 1α,24,25-(OH)₃-D₂; 1α,24,25-(OH)₃-D₄; 1α-(OH)-25-FD₃; 1α-(OH)-25-FD₄; 1α-(OH)-25-FD₂; 1α,24-(OH)₂-D₄; 1α,24-(OH)₂-D₃; 1α,24-(OH)₂-D₂; 1α,24-(OH)₂-25-FD₄; 1α,24-(OH)₂-25-FD₃; 1α,24-(OH)₂-25-FD₂; 1α,25(OH)₂-26,27-F₆-22-ene-D₃; 1α,25-(OH)₂-26,27-F₆-D₃; 1α,25S-(OH)₂-26-F₃-D₃; 1α,25-(OH)₂-24-F₂-D₃; 1α,25S,26-(OH)₂-22-ene-D₃; 1α,25R,26-(OH)₂-22-ene-D₃; 1α,25-(OH)₂-D₂; 1α,25-(OH)₂-24-epi-D₃; 1α,25-(OH)₂-23-yne-D₃; 1α,25-(OH)₂-24R-F-D₃; 1α,25S,26-(OH)₂-D₃; 1α,24R-(OH)₂-25F-D₃; 1α,25-(OH)₂-26,27-F₆-23-yne-D₃; 1α,25R-(OH)₂-26-F₃-D₃; 1α,25,28-(OH)₃-D₂; 1α,25-(OH)₂-16-ene-23-yne-D₃; 1α,24R,25-(OH)₃-D₃; 1α,25-(OH)₂-26,27-F₆-23-ene-D₃; 1α,25R-(OH)₂-22-ene-26-F₃-D₃; 1α,25S-(OH)₂-22-ene-26-F₃-D₃; 1α,25R-(OH)₂-D₃-26,26,26-d₃; 1α,25S-(OH)₂-D₃-26,26,26-d₃; and 1α,25R-(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

The term “radiotherapeutic agent,” as used herein, is intended to refer to any radiotherapeutic agent known to one of skill in the art to be effective to treat or ameliorate a hyperproliferative disorder, without limitation. For instance, the radiotherapeutic agent can be an agent such as those administered in brachytherapy or radionuclide therapy.

Brachytherapy can be administered according to any schedule, dose, or method known to one of skill in the art to be effective in the treatment or amelioration of a hyperproliferative disorder, without limitation. In general, brachytherapy comprises insertion of radioactive sources into the body of a subject to be treated for cancer, such as inside the tumor itself, such that the tumor is maximally exposed to the radioactive source, and minimizing the exposure of healthy tissue. Representative radioisotopes that can be administered in brachytherapy include, but are not limited to, phosphorus 32, cobalt 60, palladium 103, ruthenium 106, iodine 125, cesium 137, iridium 192, xenon 133, radium 226, californium 252, or gold 198. Methods of administering and apparatuses and compositions useful for brachytherapy are described in Mazeron et al., Sem. Rad. Onc. 12:95-108 (2002) and U.S. Pat. Nos. 6,319,189, 6,179,766, 6,168,777, 6,149,889, and 5,611,767.

Radionuclide therapy can be administered according to any schedule, dose, or method known to one of skill in the art to be effective in the treatment or amelioration of a hyperproliferative disorder, without limitation. In general, radionuclide therapy comprises systemic administration of a radioisotope that preferentially accumulates in or binds to the surface of cancerous cells. The preferential accumulation of the radionuclide can be mediated by a number of mechanisms, including, but not limited to, incorporation of the radionuclide into rapidly proliferating cells, specific accumulation of the radionuclide by the cancerous tissue without special targeting, or conjugation of the radionuclide to a biomolecule specific for a neoplasm.

Representative radioisotopes that can be administered in radionuclide therapy include, but are not limited to, phosphorus 32, yttrium 90, dysprosium 165, indium 111, strontium 89, samarium 153, rhenium 186, iodine 131, iodine 125, lutetium 177, and bismuth 213. While all of these radioisotopes may be linked to a biomolecule providing specificity of targeting, iodine 131, indium 111, phosphorus 32, samarium 153, and rhenium 186 may be administered systemically without such conjugation. One of skill in the art may select a specific biomolecule for use in targeting a particular neoplasm for radionuclide therapy based upon the cell-surface molecules present on that neoplasm. Examples of biomolecules providing specificity for particular cell are reviewed in an article by Thomas, Cancer Biother. Radiopharm. 17:71-82 (2002), which is incorporated herein by reference in its entirety. Furthermore, methods of administering and compositions useful for radionuclide therapy may be found in U.S. Pat. Nos. 6,426,400, 6,358,194, 5,766,571.

The term “radiotherapeutic treatment,” as used herein, is intended to refer to any radiotherapeutic treatment known to one of skill in the art to be effective to treat or ameliorate a hyperproliferative disorder, without limitation. For instance, the radiotherapeutic treatment can be external-beam radiation therapy, thermotherapy, radiosurgery, charged-particle radiotherapy, neutron radiotherapy, or photodynamic therapy.

External-beam radiation therapy can be administered according to any schedule, dose, or method known to one of skill in the art to be effective in the treatment or amelioration of a hyperproliferative disorder, without limitation. In general, external-beam radiation therapy comprises irradiating a defined volume within a subject with a high energy beam, thereby causing cell death within that volume. The irradiated volume preferably contains the entire cancer to be treated, and preferably contains as little healthy tissue as possible. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652.

Thermotherapy can be administered according to any schedule, dose, or method known to one of skill in the art to be effective in the treatment or amelioration of a hyperproliferative disorder, without limitation. In certain embodiments, the thermotherapy can be cryoablation therapy. In other embodiments, the thermotherapy can be hyperthermic therapy. In still other embodiments, the thermotherapy can be a therapy that elevates the temperature of the tumor higher than in hyperthermic therapy.

Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al., Sem. Urol. Oncol. 19:133-140 (2001) and U.S. Pat. Nos. 6,383,181, 6,383,180, 5,993,444, 5,654,279, 5,437,673, and 5,147,355.

Hyperthermic therapy typically involves elevating the temperature of a neoplastic mass to a range from about 42° C. to about 44° C. The temperature of the cancer may be further elevated above this range; however, such temperatures can increase injury to surrounding healthy tissue while not causing increased cell death within the tumor to be treated. The tumor may be heated in hyperthermic therapy by any means known to one of skill in the art without limitation. For example, and not by way of limitation, the tumor may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radiowaves can be generated by waveguide applicators, horn, spiral, current sheet, and compact applicators.

Other methods, apparatuses and compositions for raising the temperature of a tumor are reviewed in an article by Wust et al., Lancet Oncol. 3:487-97 (2002), and described in U.S. Pat. Nos. 6,470,217, 6,379,347, 6,165,440, 6,163,726, 6,099,554, 6,009,351, 5,776,175, 5,707,401, 5,658,234, 5,620,479, 5,549,639, and 5,523,058.

Radiosurgery can be administered according to any schedule, dose, or method known to one of skill in the art to be effective in the treatment or amelioration of a hyperproliferative disorder, without limitation. In general, radiosurgery comprises exposing a defined volume within a subject to a manually directed radioactive source, thereby causing cell death within that volume. The irradiated volume preferably contains the entire cancer to be treated, and preferably contains as little healthy tissue as possible. Typically, the tissue to be treated is first exposed using conventional surgical techniques, then the radioactive source is manually directed to that area by a surgeon. Alternatively, the radioactive source can be placed near the tissue to be irradiated using, for example, a laparoscope. Methods and apparatuses useful for radiosurgery are further described in Valentini et al., Eur. J. Surg. Oncol. 28:180-185 (2002) and in U.S. Pat. Nos. 6,421,416, 6,248,056, and 5,547,454.

Charged-particle radiotherapy can be administered according to any schedule, dose, or method known to one of skill in the art to be effective in the treatment or amelioration of a hyperproliferative disorder, without limitation. In certain embodiments, the charged-particle radiotherapy can be proton beam radiotherapy. In other embodiments, the charged-particle radiotherapy can be helium ion radiotherapy. In general, charged-particle radiotherapy comprises irradiating a defined volume within a subject with a charged-particle beam, thereby causing cellular death within that volume. The irradiated volume preferably contains the entire cancer to be treated, and preferably contains as little healthy tissue as possible. A method for administering charged-particle radiotherapy is described in U.S. Pat. No. 5,668,371.

Neutron radiotherapy can be administered according to any schedule, dose, or method known to one of skill in the art to be effective in the treatment or amelioration of a hyperproliferative disorder, without limitation. In certain embodiments, the neutron radiotherapy can be a neutron capture therapy. In such embodiments, a compound that emits radiation when bombarded with neutrons and preferentially accumulates in a neoplastic mass is administered to a subject. Subsequently, the tumor is irradiated with a low energy neutron beam, activating the compound and causing it to emit decay products that kill the cancerous cells. The compound to be activated can be caused to preferentially accumulate in the target tissue according to any of the methods useful for targeting of radionuclides, as described above, or in the methods described in Laramore, Semin. Oncol. 24:672-685 (1997) and in U.S. Pat. Nos. 6,400,796, 5,877,165, 5,872,107, and 5,653,957.

In other embodiments, the neutron radiotherapy can be a fast neutron radiotherapy. In general, fast neutron radiotherapy comprises irradiating a defined volume within a subject with a neutron beam, thereby causing cellular death within that volume.

Photodynamic therapy can be administered according to any schedule, dose, or method known to one of skill in the art to be effective in the treatment or amelioration of cancer, without limitation. In general, photodynamic therapy comprises administering a photosensitizing agent that preferentially accumulates in a neoplastic mass and sensitizes the neoplasm to light, then exposing the tumor to light of an appropriate wavelength. Upon such exposure, the photosensitizing agent catalyzes the production of a cytotoxic agent, such as, e.g., singlet oxygen, which kills the cancerous cells. Methods of administering and apparatuses and compositions useful for photodynamic therapy are disclosed in Hopper, Lancet Oncol. 1:212-219 (2000) and U.S. Pat. Nos. 6,283,957, 6,071,908, 6,011,563, 5,855,595, 5,716,595, and 5,707,401.

Radiotherapy can be administered to destroy hyperproliferative cells before or after surgery, before or after chemotherapy, and sometimes during chemotherapy. Radiotherapy may also be administered for palliative reasons to relieve symptoms of a hyperproliferative disorder, for example, to lessen pain. Among the types of tumors that can be treated using radiotherapy are localized tumors that cannot be excised completely and metastases and tumors whose complete excision would cause unacceptable functional or cosmetic defects or be associated with unacceptable surgical risks.

It will be appreciated that both the particular radiation dose to be utilized in treating a hyperproliferative disorder and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980. In certain embodiments, radiotherapeutic agents and treatments may be administered at doses lower than those known in the art due to the additive or synergistic effect of the compound having Formula I.

Compositions in accordance with the present invention may be employed for administration in any appropriate manner, e.g., oral or buccal administration, e.g., in unit dosage form, for example in the form of a tablet, in a solution, in hard or soft encapsulated form including gelatin encapsulated form, sachet, or lozenge. Compositions may also be administered parenterally or topically, e.g., for application to the skin, for example in the form of a cream, paste, lotion, gel, ointment, poultice, cataplasm, plaster, dermal patch or the like, or for ophthalmic application, for example in the form of an eye-drop, -lotion or -gel formulation. Readily flowable forms, for example solutions, emulsions and suspensions, may also be employed e.g., for intralesional injection, or may be administered rectally, e.g., as an enema or suppository, or intranasal administration, e.g., as a nasal spray or aerosol. Microcrystalline powders may be formulated for inhalation, e.g., delivery to the nose, sinus, throat or lungs. Transdermal compositions/devices and pessaries may also be employed for delivery of the compounds of the invention. The compositions may additionally contain agents that enhance the delivery of the compounds having Formula I (or other active agents), e.g., liposomes, polymers or co-polymers (e.g., branched chain polymers). Preferred dosage forms of the present invention include oral dosage forms and intravenous dosage forms.

Intravenous forms include, but are not limited to, bolus and drip injections. In preferred embodiments, the intravenous dosage forms are sterile or capable of being sterilized prior to administration to a subject since they typically bypass the subject's natural defenses against contaminants. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

The pharmaceutical compositions of the present invention may further comprise one or more additives. Additives that are well known in the art include, e.g., detackifiers, anti-foaming agents, buffering agents, antioxidants (e.g., ascorbic acid, ascorbyl palmitate, sodium ascorbate, butylated hydroxyanisole (BHA), butylated hydroxytoluene (BHT), propyl gallate, malic acid, fumaric acid, potassium metabisulfite, sodium bisulfite, sodium metabisulfite, and tocopherols, e.g., α-tocopherol (vitamin E)), preservatives, chelating agents, viscomodulators, tonicifiers, flavorants, colorants, odorants, opacifiers, suspending agents, binders, fillers, plasticizers, lubricants, and mixtures thereof. The amounts of such additives can be readily determined by one skilled in the art, according to the particular properties desired, and can be formulated such that compounds having Formula I are stable, e.g., not reduced by antioxidant additives.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents may not be required. Use of thickening agents is, on the other hand, indicated, e.g., where topical application is foreseen.

In one embodiment of the invention, compounds having Formula I are formulated as described in WO 03/076387. In particular, the compounds are formulated such that upon dissolution in aqueous solution the pH of the solution is in the range of 5 to 9.

When an active vitamin D compound is used in the practice of the invention, a pharmaceutical composition is provided comprising (a) a lipophilic phase component, (b) one or more surfactants, (c) an active vitamin D compound; wherein said composition is an emulsion pre-concentrate, which upon dilution with water, in a water to composition ratio of about 1:1 or more of said water, forms an emulsion having an absorbance of greater than 0.3 at 400 nm. The pharmaceutical composition of the invention may further comprise a hydrophilic phase component. Such pharmaceutical compositions are described in PCT International Application Publication No. WO 03/047595.

In certain aspects of the invention, a pharmaceutical composition may comprise an active vitamin D compound, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In other embodiments, the pharmaceutical compositions comprise 50% MIGLYOL 812 and 50% vitamin E TPGS, 90% MIGLYOL 812 and 10% vitamin E TPGS, or 95% MIGLYOL 812 and 5% vitamin E TPGS.

In another embodiment of the invention, the pharmaceutical compositions comprise an active vitamin D compound and a lipophilic component, e.g., around 100% MIGLYOL 812.

In a preferred embodiment, the pharmaceutical compositions comprise 50% MIGLYOL 812, 50% vitamin E TPGS, and small amounts of BHA and BHT. This formulation has been shown to be unexpectedly stable, both chemically and physically. The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound, thereby enabling high dose pulse administration of active vitamin D compounds for the treatment of hyperproliferative disorders.

In certain embodiments, the pharmaceutical compositions comprising an active vitamin D compound further comprise a compound having Formula I.

Although the dosage of the compound having Formula I will vary according to the activity and/or toxicity of the particular compound, the condition being treated, and the physical form of the pharmaceutical composition being employed for administration, it may be stated by way of guidance that a dosage selected in the range from 0.1 to 20 mg/kg of body weight per day will often be suitable, although higher dosages, such as 0.1 to 50 mg/kg of body weight per day may be useful. Those of ordinary skill in the art are familiar with methods for determining the appropriate dosage. Methods for assessing the toxicity, activity and/or selectivity of the compounds having Formula I may be carried out as described in Lee et al., supra, and PCT Published International Application WO 92/15300, supra, and may be useful for approximating and/or determining dose ranges for compounds having Formula I.

In certain instances, the dosage of the compounds having Formula I will be lower, e.g., when used in combination with at least a second hyperproliferative disorder treatment, and may vary according to the activity and/or toxicity of the particular compound, the condition being treated, and the physical form of the pharmaceutical composition being employed for administration.

When the composition of the present invention is formulated in unit dosage form, the compound having Formula I will preferably be present in an amount of between 0.1 and 200 μg per unit dose. More preferably, the amount of compound having Formula I per unit dose will be about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μg or any amount therein.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative individually inventive teachings only and not as not limiting the invention in its broadest aspect.

The amount of compound having Formula I in compositions of the invention will of course vary, e.g., depending on the intended route of administration and to what extent other components are present. In general, however, the compound having Formula I will suitably be present in an amount of from about 0.005% to 20% by weight based upon the total weight of the composition. In certain embodiments, the compound having Formula I is present in an amount of from about 0.01% to 15% by weight based upon the total weight of the composition.

In addition to the foregoing, the present invention also provides a process for the production of a pharmaceutical composition as hereinbefore defined, which process comprises bringing the individual components thereof into intimate admixture and, when required, compounding the obtained composition in unit dosage form, for example filling said composition into tablets, gelatin, e.g., soft or hard gelatin, capsules, or non-gelatin capsules.

Compounds having Formula I can be prepared by methods well known in the art and as illustrated by exemplary reactions in the following Schemes.

Scheme 1: Route to 2,3-difluoro-AQ4N (5); 2,3,6-trifluoro-AQ4N (7) and 2,3,6,7-tetrafluoro-AQ4N (8)

1,2,3,4-Tetrafluoro-5,8-dihydroxyanthraquinone (2) is available from Aldrich Co. Selective displacement of the reactive fluorines at positions 1 and 4 with N,N-dimethylethylenediamine at room temperature in pyridine according to the method of Lee and Denny (J. Chem. Soc. Perkin Trans. I, 2755 (1999)) will give tetramine (4). Selective N-oxidation of the two tertiary amine groups with m-chloroperoxybenzoic acid (MCPBA) in CH₂Cl₂ as per Lee and Denny will give 2,3-difluoro-AQ4N (5). Monofluorination of 5 with N-fluoropyridinium triflate (6) (Umemoto et al., Org. Synth. Coll. Vol. 8, 286) will give 2,3,6-trifluoro-AQ4N (7). Addition of another equivalent of fluorination agent 6 will give 2,3,6,7-tetrafluoro-AQ4N (8).

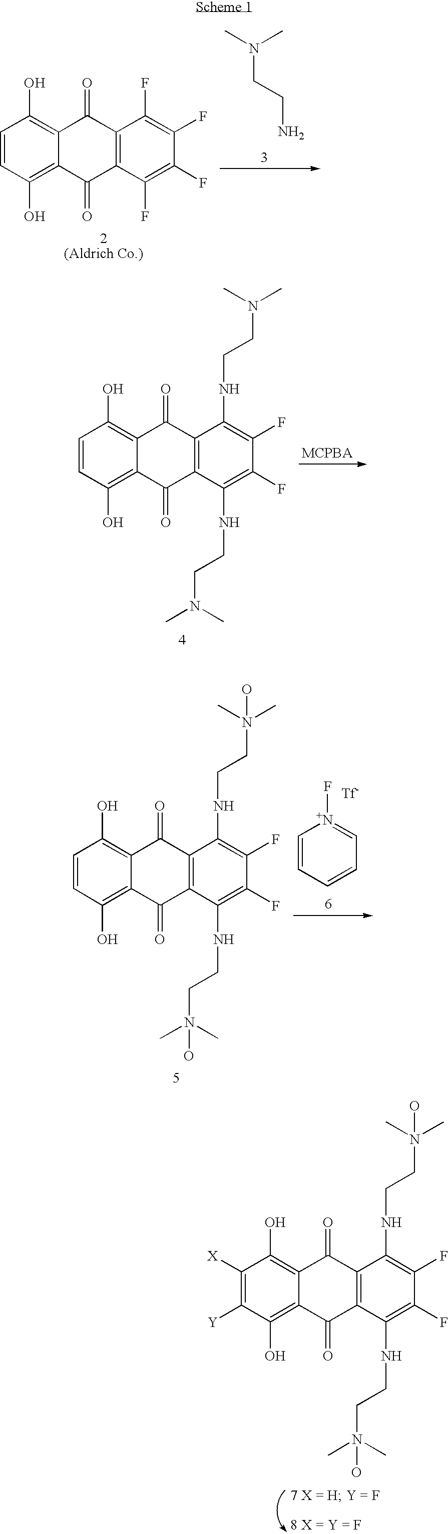

An alternative route to (7) and (8) parallels the route shown below in Scheme 2 for the conversion of (9) to (14). Fluorohydroquinones 9 and 10 are allowed to react with tetrafluorophthalic anhydride in place of difluorophthalic anhydride (11), leading after several steps to (7) and (8), respectively.

Scheme 2: Route to 6-fluoro-AQ4N (14) and 6,7-difluoro-AQ4N (15)

Fluorohydroquinone (9) (Feiring et al., J. Org. Chem. 40:2543 (1975)) treated with difluorophthalic anhydride (11) as per Lee and Denny will give trifluoroanthraquinone (12). Displacement of the two reactive fluorine atoms in (12) with diamine (3) as per Lee and Denny will give tetramine (13), oxidation of which with MCPBA will give 6-fluoro-AQ4N (14). Selective fluorination of (14) on the reactive hydroquinone ring using fluorinating reagent 6 will give 6,7-difluoro-AQ4N (15). If there is competitive fluorination on the diamine-bearing ring, then the fluorination may be performed under mildly basic conditions, taking advantage of the enhanced reactivity expected of the hydroquinone portion of the molecule toward halogenation when in its anionic form. A third alternative route to (15) begins with commercially available 2,3-difluorohydroquinone (10) (SYNTHON Chemicals GmbH & Co. KG) and will follow a route analogous to that of (9) to (14).

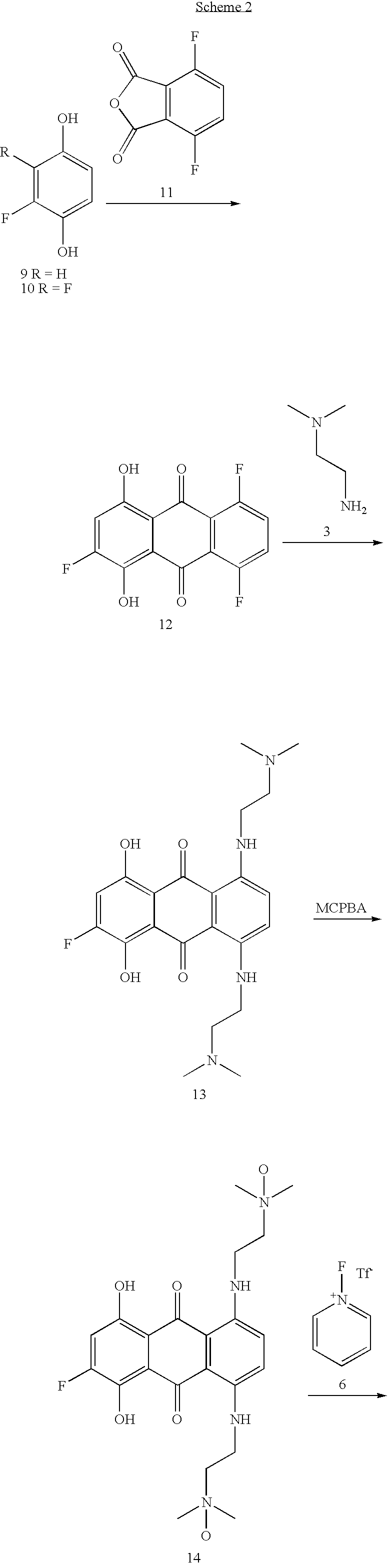

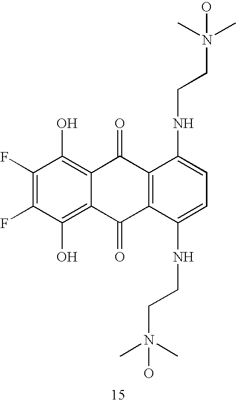

### Scheme 3: Route to N-alkylated Versions (19)-(24) of AQ4N

Difluoroanthraquinone (16) treated with N,N,N′-trimethylethylenediamine (17) according to Lee and Denny will give tetramine (18). MCPBA oxidation of (18) will give N,N′-dimethylated AQ4N (19). It will be apparent to those skilled in the art that AQ4N analogs (20)-(24) may be prepared in a similar fashion by using N,N,N′-trimethylethylenediamine (17) in place of N,N-dimethylethylenediamine (3) in Schemes 1 and 2. Also, other N,N-dimethyl-N′-alkylated ethylenediamines may be used in place of (17), for example, the commercially available secondary amine, 2-{[2-(diethylamino)ethyl]amino}ethanol. In the latter case, (19a) will be the AQ4N derivative that will be synthesized using the chemistry shown in Scheme 3.

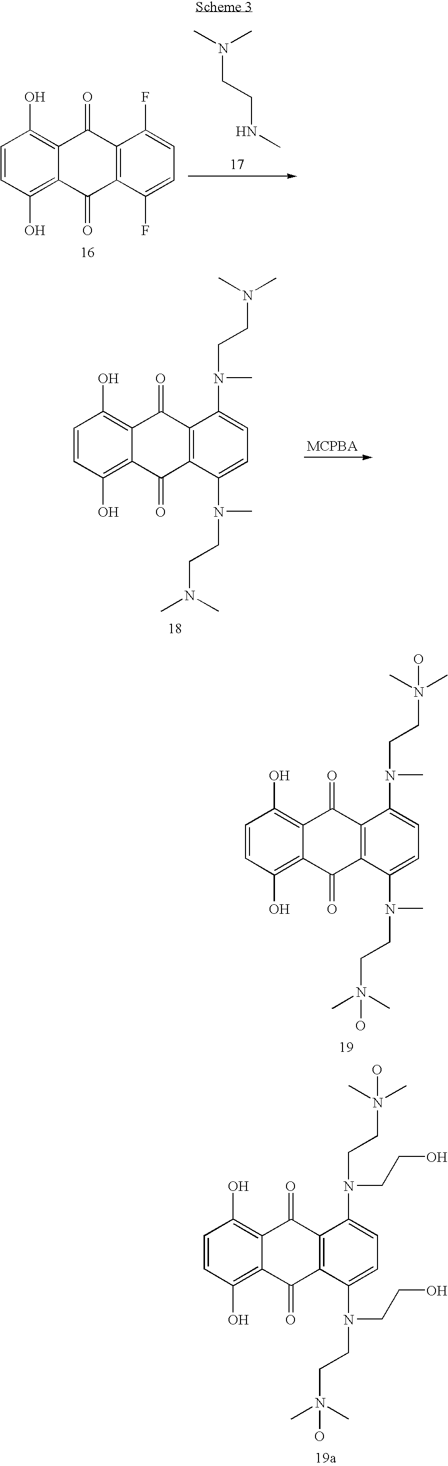

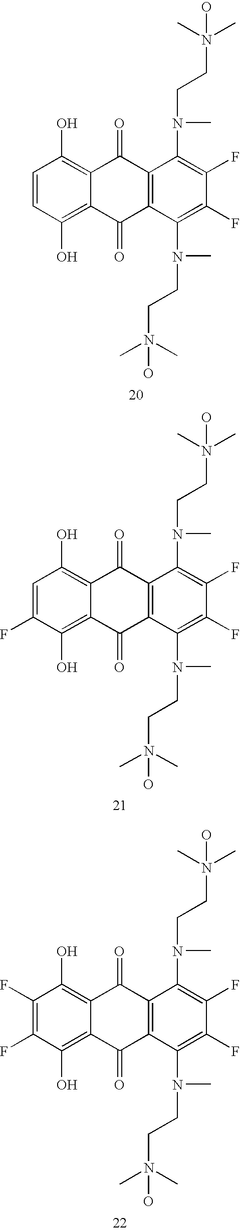

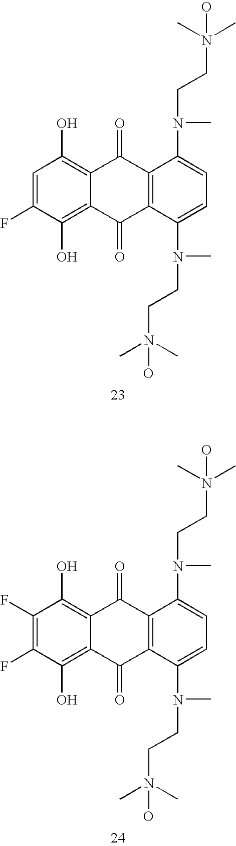

Scheme 4: Route to 2-fluoro-AQ4N (31) and 2,6,7-trifluoro-AQ4N (32) and Some Derivatives

Scheme 4 provides a synthetic route to a family of 2-fluorinated AQ4N derivatives. For example, 2,3,5-trifluorophthalic anhydride (EP 514863 A2) reacted separately with hydroquinones (10) or (25) as per Lee and Denny, will afford fluorinated anthraquinones (27) and (28), respectively. Reaction of either with diamine (3) will proceed selectively with displacement of the highly reactive fluorine atoms at positions 1 and 4 and will give tetramines (29) and (30), respectively. Oxidation with MCPBA will give 2,6,7-trifluoro-AQ4N (31) and 2-fluoro-AQ4N (32), respectively. Fluoride (32) may undergo nucleophilic aromatic substitution (Smith et al., Advanced Organic Chemistry, Fifth Ed., Wiley, New York, 2001, pp. 850-893) by displacement of the fluorine atom at position 3 under somewhat more vigorous conditions with a variety of nucleophiles, as will be apparent to those skilled in the art. For example, propargyl alcohol (Levin et al., Synthetic Commun. 32:1401 (2002)) will react with (32) under basic conditions to give propargyl ether (32a).

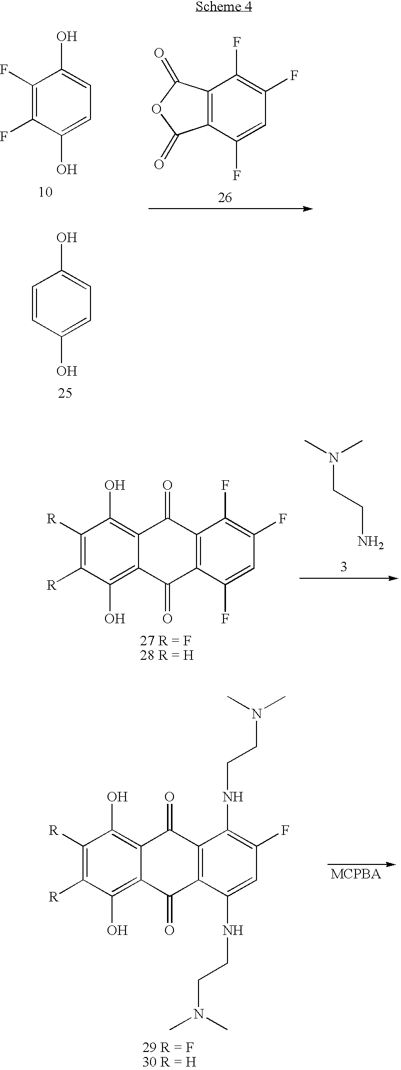

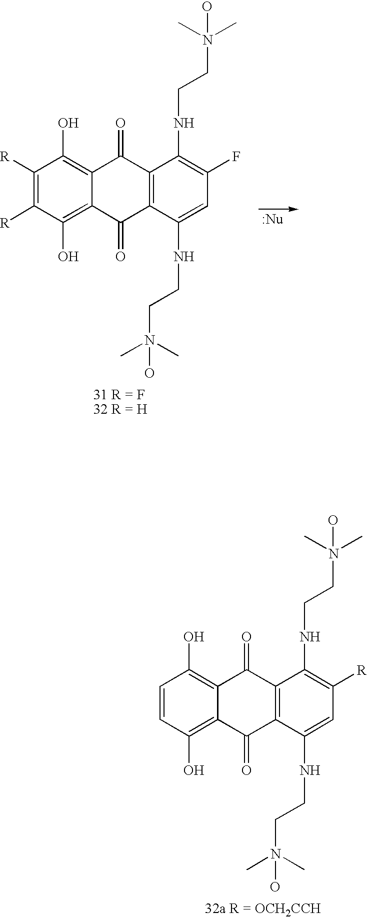

Scheme 5: Route to 2-chloro-Substituted AQ4N (38)-(47) and 2-alkynyl AQ4N (51)

Monochlorination of difluorophthalic anhydride (11) will give chlorodifluorophthalic anhydride (33), which will be reacted with hydroquinone (25) as per Lee and Denny to give (34). Reaction of (34) with diamine (3) will give tetramine (36), oxidation of which with MCPBA will give 2-chloro-AQ4N (38). The compound (25) may be replaced by a substituted hydroquinone such as (10). Also, other N,N-dimethyl-N′-alkylated ethylenediamines may be used in place of (3), for example, diamine (17) or the commercially available secondary amine, 2-{[2-(diethylamino)ethyl]amino}ethanol. Therefore, 2-chloro-AQ4N derivatives (40), (42), (44) and (46), for example, will readily be prepared from (33) by the route shown in Scheme 5.

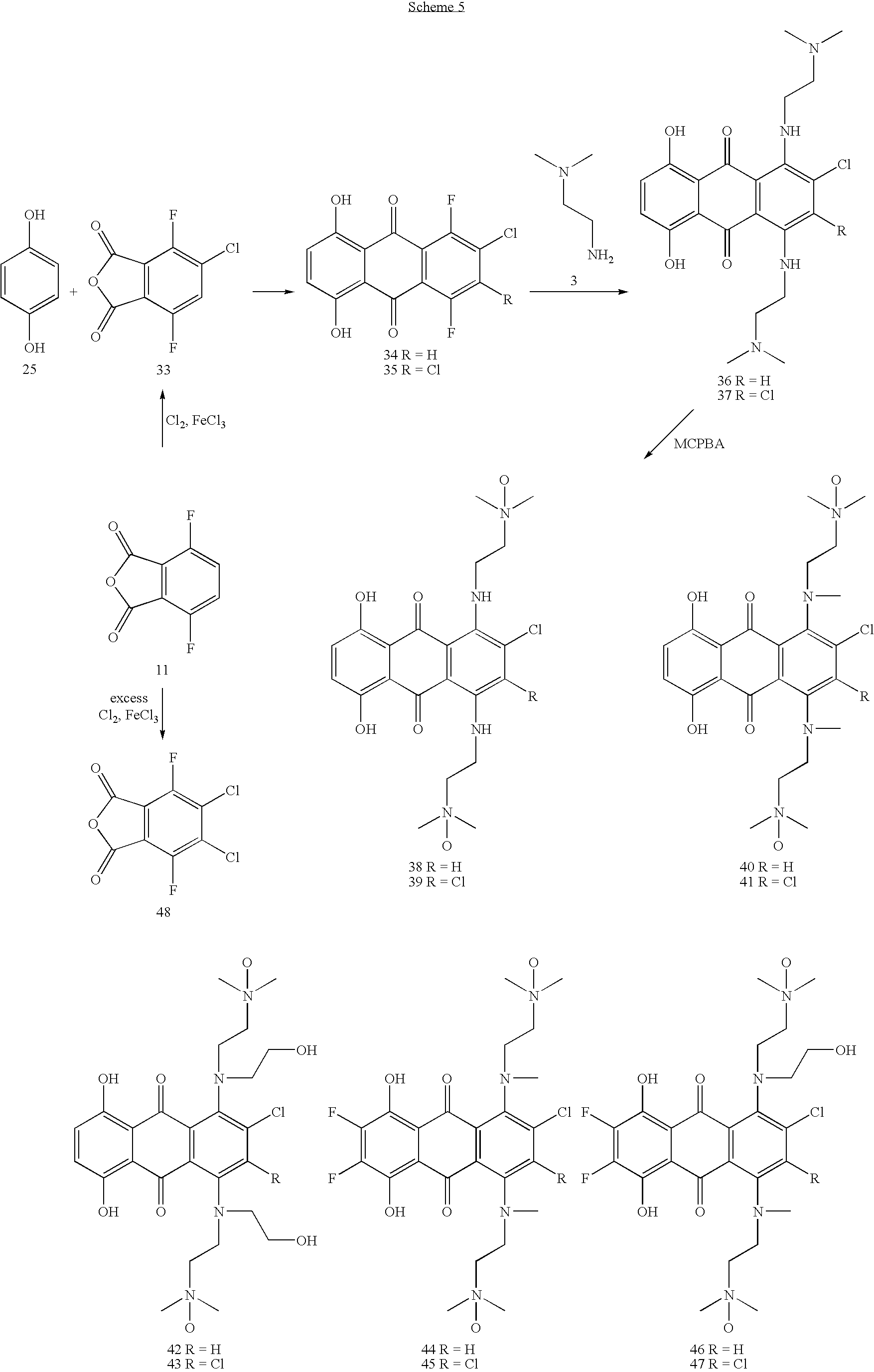

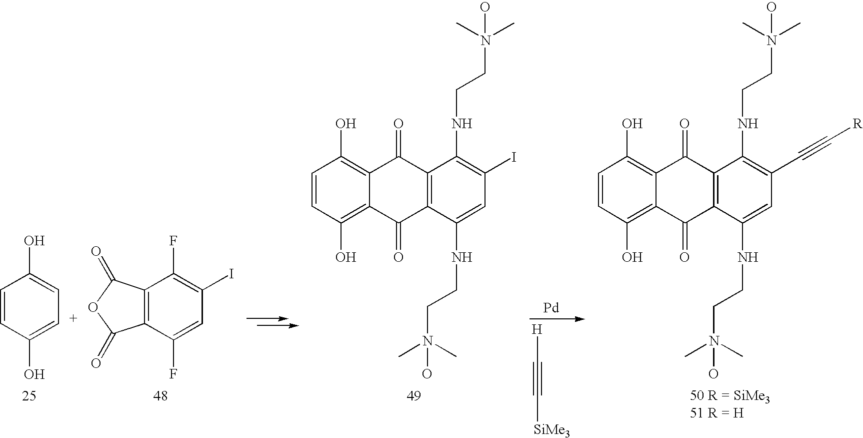

Dichlorination of phthalic anhydride (11) will produce tetrahalophthalic anhydride (48). The compound (48) may be used in place of (33) for the syntheses shown in Scheme 5. In that event, 2,3-dichloro-AQ4N derivatives (39), (41), (43), (45) and (47) are examples of halogenated AQ4N derivatives that will be obtained by the chemistry shown in Scheme 5. 2-Brominated and 2,3-dibrominated versions of these AQ4N derivatives will be prepared by using versions of (33) and (48) in which bromine replaces chlorine. If a version of (33) is used in which the chlorine atom is replaced by iodine, then a series of 2-iodinated AQ4N molecules as exemplified by (49) will be prepared. Each of these will be used in a Pd-catalyzed Sonogashira coupling reaction with a variety of terminal alkynes, for example, trimethylsilylacetylene, to prepare a series of 2-alkynyl AQ4N molecules as illustrated by the synthesis of alkyne (50). Treatment of (50) with Bu₄NF will give terminal alkyne (51). Further chemical modifications will be made on the alkyne unit of (51) leading to other 2-substituted AQ4N molecules.

### Scheme 6: Route to Azetidinyl- and Pyrrolidino-N-Oxide Analogs of AQ4N

Scheme 6 illustrates general routes to AQ4N analogs in which the two terminal N,N-dimethylamine N-oxide moieties of AQ4N are replaced by rings, for example, azetidine- and pyrrolidine N-oxides. N-Alkylazetidine N-oxides are stable compounds.

For example, reaction of (16) with N-(2-aminoethyl)azetidine (53) as per Lee and Denny will give tetramine (54). Oxidation with MCPBA will give bis N-oxide (56). Other N-(2-aminoethyl) heterocyclic amines may be used in place of (52), for example, (53). N-(2-alkyl- or 2-(substituted alkyl))ethyl heterocyclic amines of the formula (58) will be used in place of (52). Additionally, anthraquinone (16) will be replaced by other anthraquinones, for example, (2), (12), (27), (28), (34), and (35) leading to a series of AQ4N analogs, for example, (59)-(64).

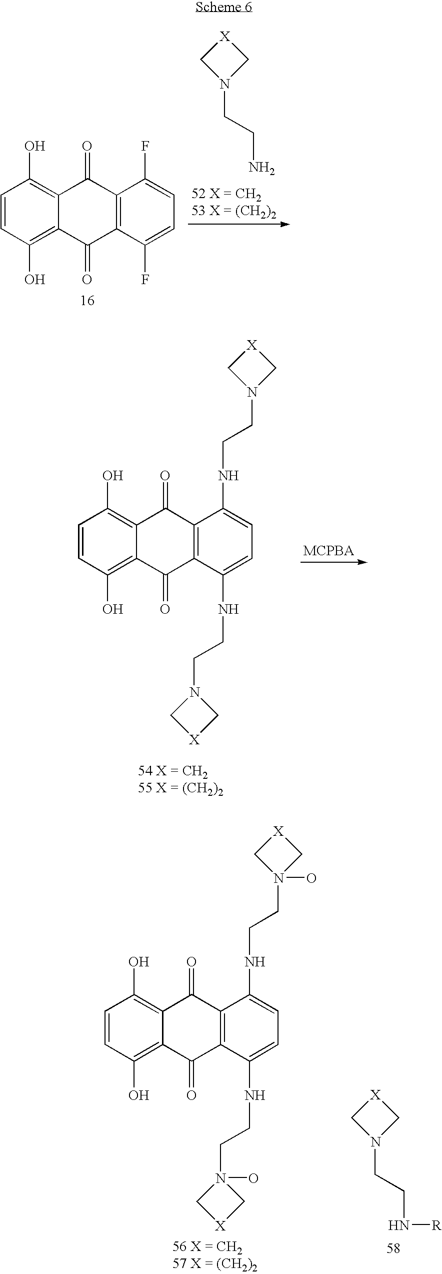

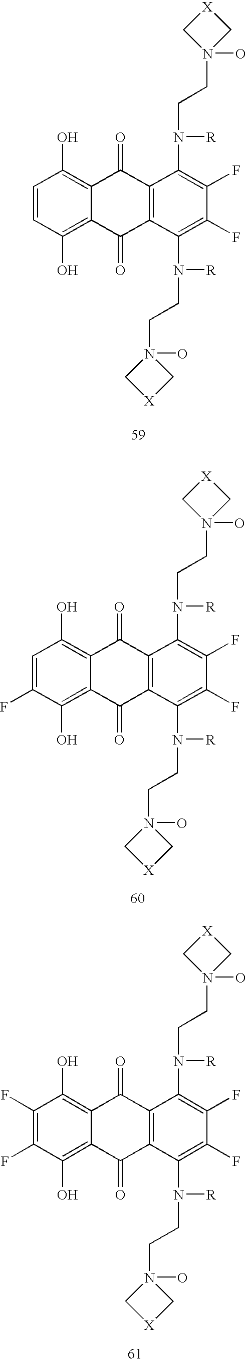

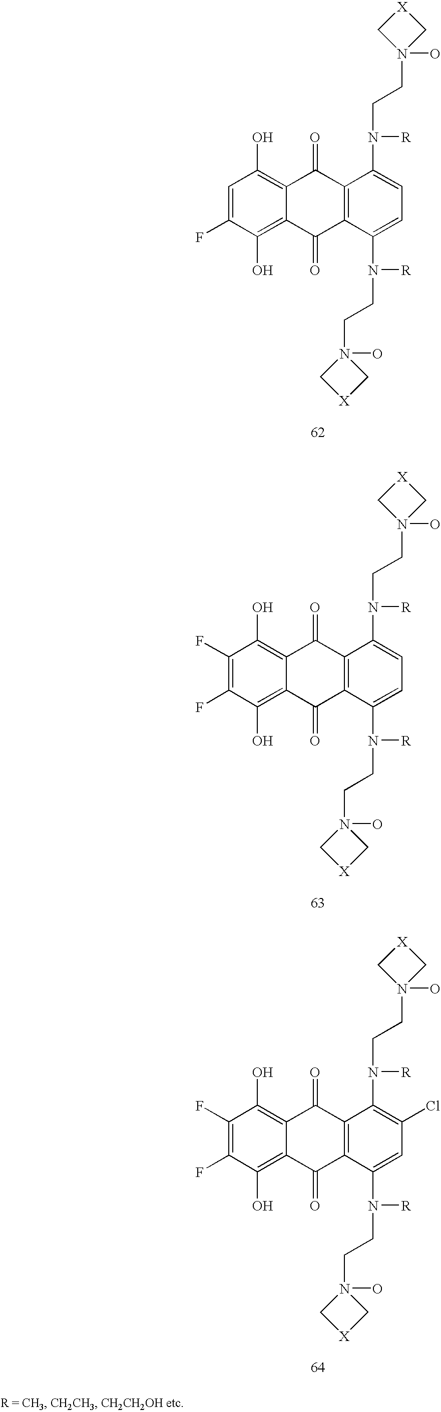

### Schemes 7 and 8: Route to Other 2-Substituted and 2,3-Disubstituted Analogs of AQ4N

Commercially available 3,6-difluorophthalic anhydride (11) will serve as a precursor for a family of 4-substituted 3,6-difluorophthalic anhydrides as shown in Scheme 7. Commercially available 3,6-dichlorophthalic anhydride will also be used in place of (11) if the fluorine atoms in the phthalic anhydride derivatives become too reactive toward nucleophilic displacement. While the anhydride forms are shown in Scheme 7, parallel chemistry will be performed in many cases on the corresponding phthalic acid should the anhydride become hydrolyzed. Reconversion of the diacid into the anhydride will be effected by heating the diacid. Specifically, chlorosulfonation of (11) with chlorosulfonic acid will lead to sulfonyl chloride (66). This compound will be reacted with a variety of alcohols or amines to give the corresponding sulfonyl derivative, for example, (65).

Nitration of (11) will give (67), which upon reduction and acylation will give a series of amides exemplified by (68). Commercially available 2,5-dichlorotrimellitic anhydride (69) will serve as a precursor for several 4-carbonyl-3,6-dichlorophthalic anhydrides, for example, esters (71) and amides (72) via the acid chloride (70).

The family of 2,5-difluorobenzoic acids exemplified by general structure (73) will serve as a precursor for a family of 3,6-difluorophthalic anhydrides exemplified by general structure (76). The key step will be the ortho-metallation of N,N-diethylamide (74) according to the method of Krapcho et al. (Synthetic Commun. 20:2139 (1990)). Conversion of (75) into phthalic anhydride (76) will be straightforward. A specific illustration of the method begins with commercially available benzoic acid (77), which will be converted into the diethylamide (78), metallated and carboxylated which will give (79) and cyclized which will give phthalic anhydride (80). Using an analogous sequence of reactions, one may prepare various phthalic anhydrides, for example, (76) Y=H; X=OMe, OEt, OCH₂CO₂CH₃, OCH₂CH₂OCH₂CH₂OR and many others. A wide range of 4-substituted and 4,5-disubstituted 3,6-dichloro- and 3,6-difluorophthalic anhydrides will be accessible by the chemistry shown in Scheme 7.

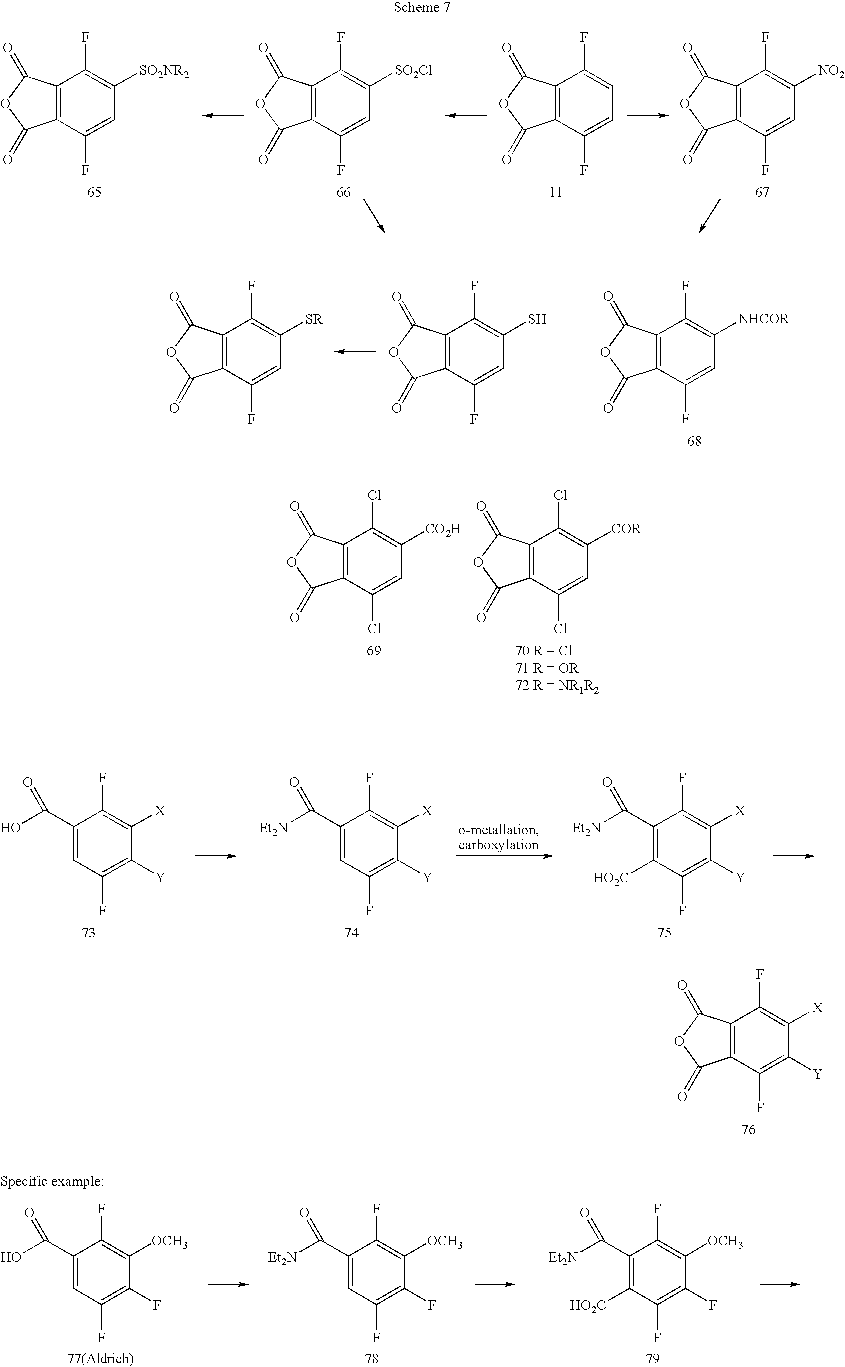

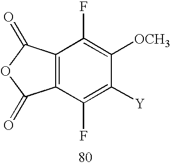

Scheme 8 shows the generalized synthesis of a series of AQ4N analogs which will begin with the reaction of 2,3-disubstituted hydroquinone (81) with 4,5-disubstituted-3,6-difluorophthalic anhydride (76) as per Lee and Denny. Use of diamines other than (3), for example, (52) or (53), will lead to a family of AQ4N analogs represented by (85).

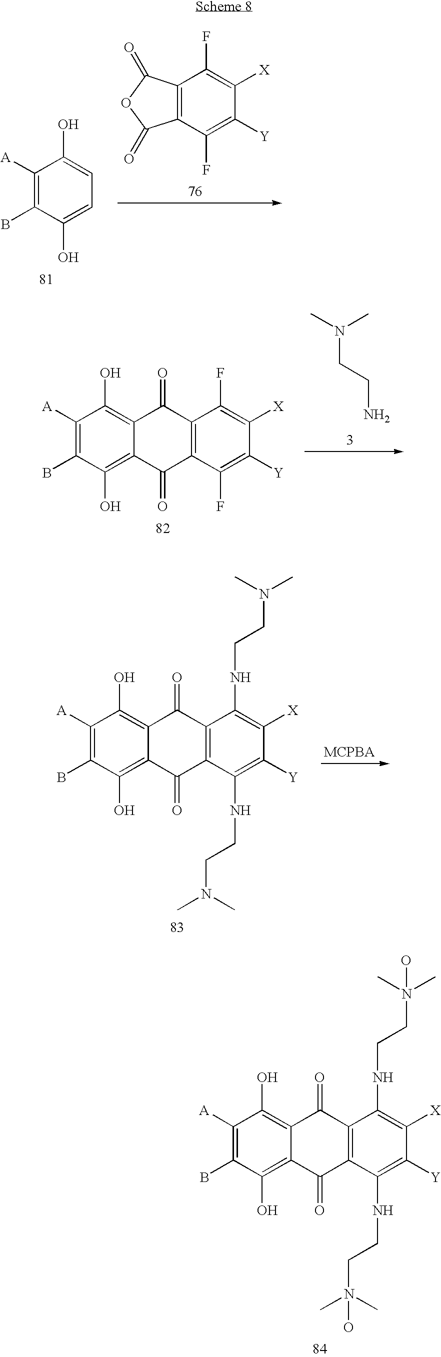

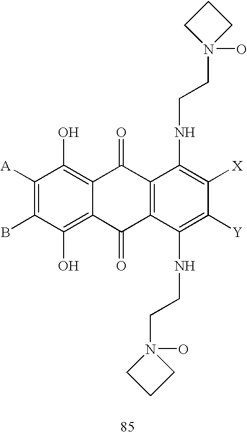

### Schemes 9-14: Route to Analogs of AQ4N Containing Additional Rings

Replacement of diamine (3) in Scheme 8 by a heterocyclic diamine as exemplified by (86) (Z=CH₂, CH₂CH₂, etc.) or alternatively, a heterocyclic amide in its anionic form as exemplified by (87) (Z=CH₂, CH₂CH₂, etc.) will lead to a series of analogs (89) or (90) of AQ4N with side chains that are more rigid than the diamine units in AQ4N. Examples are shown in Scheme 9.

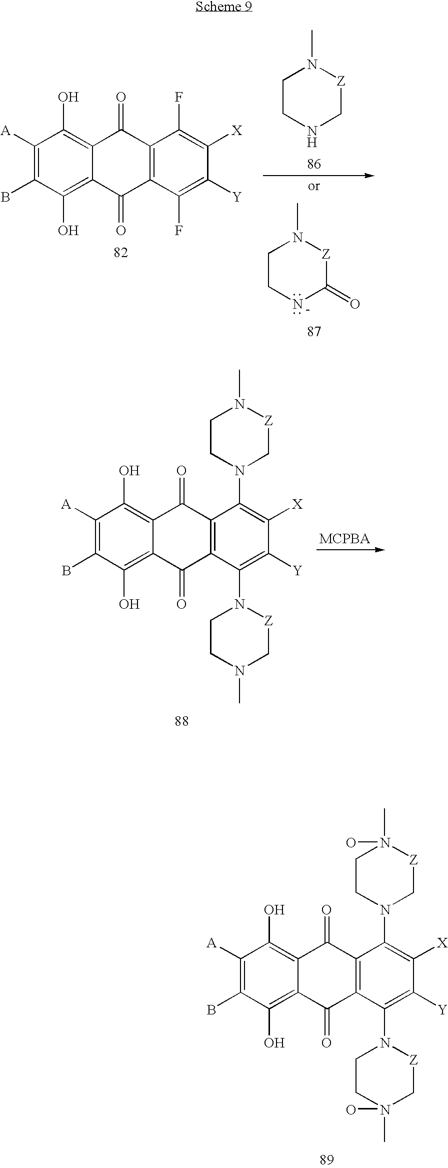

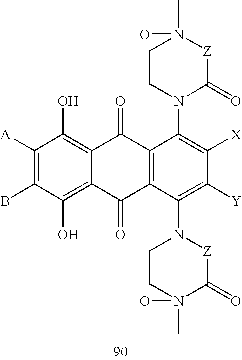

Another variation leading to a more rigid diamine unit than that in AQ4N itself will be obtained by replacing diamine (3) in Scheme 8 with a cis or trans 1,2-diaminocycloalkane as exemplified by diamine (91) (Z=CH₂, CH₂CH₂, CH₂O, etc.) in Scheme 10. The trans 1,2-diamines exist in either of two enantiomeric forms, either of which may be used in the synthesis. Representative 1,2-diamino cyclic compounds include (95)-(99).

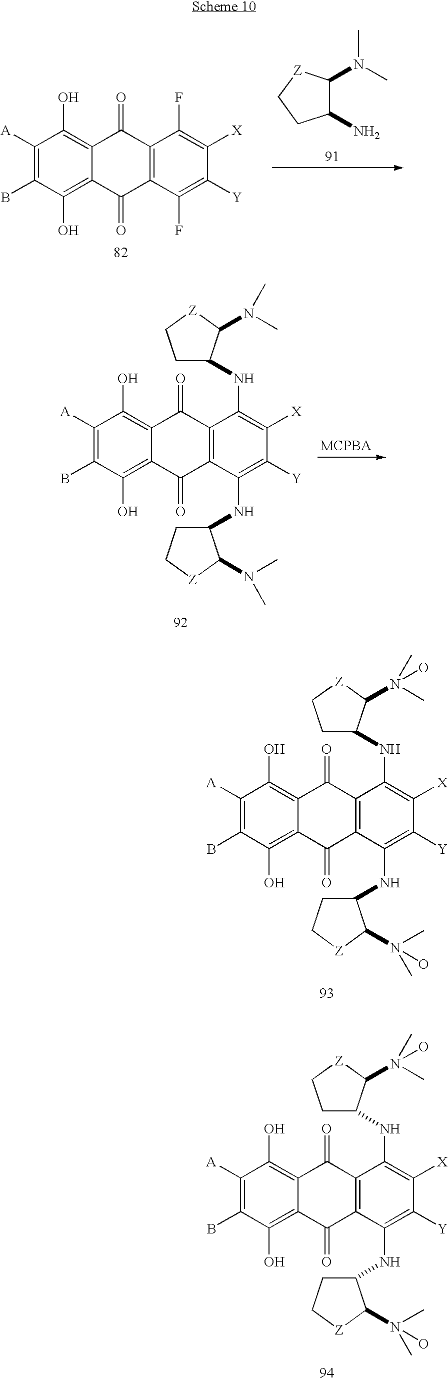

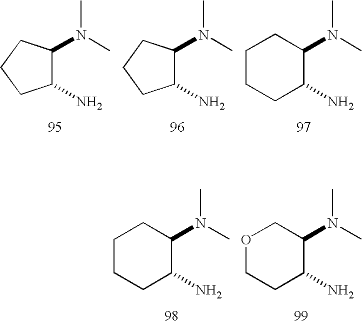

A further variation leading to a more rigid diamine unit than that in AQ4N itself will be obtained by fusing a piperazine ring to the C1-C2 bond in AQ4N as shown in Scheme 11. Cheng and others demonstrated that heating Mitoxanthrone (100) in methanolic KOH gave tetracyclic analog (101) in 82% yield. (Cheng, Synthetic Commun. 26:3929 (1996) and references cited therein). In an adaptation of Cheng's chemistry, (16) will be treated with 1 equiv of triamine (102) to give anthraquinone (103). Reaction of (103) with one equivalent of diamine (3) will give anthraquinone (104). Catalytic debenzylation with H₂/Pd will give pentaamine (105). Heating (105) in methanolic KOH will give pentaamine (106). Oxidation of the two terminal tertiary amine groups with MCPBA will give AQ4N analog (107). Analog (108) will be prepared analogously by using two equiv. of triamine (102) in the reaction with difluoride (16).

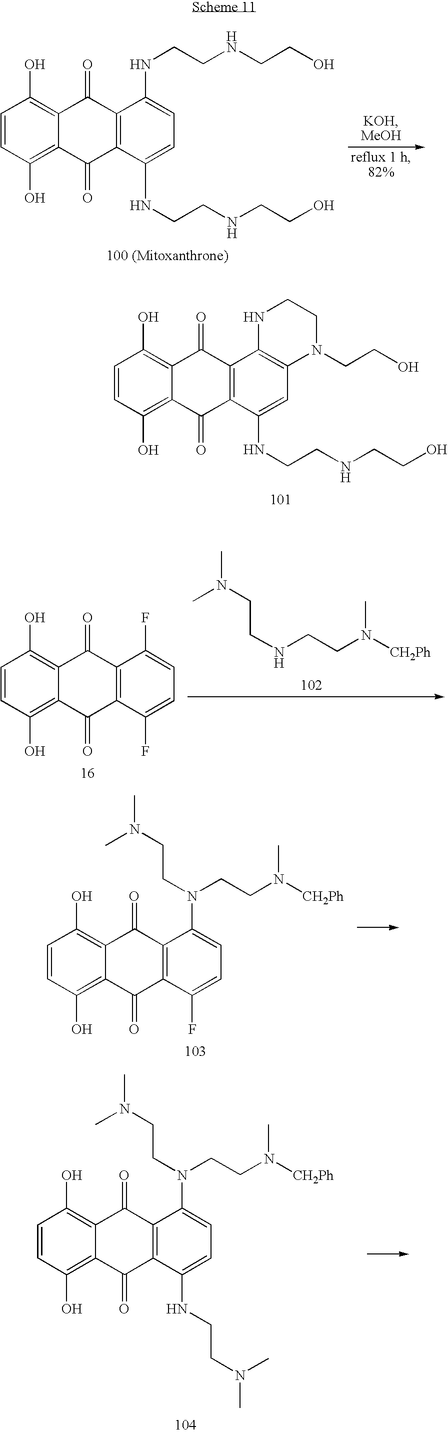

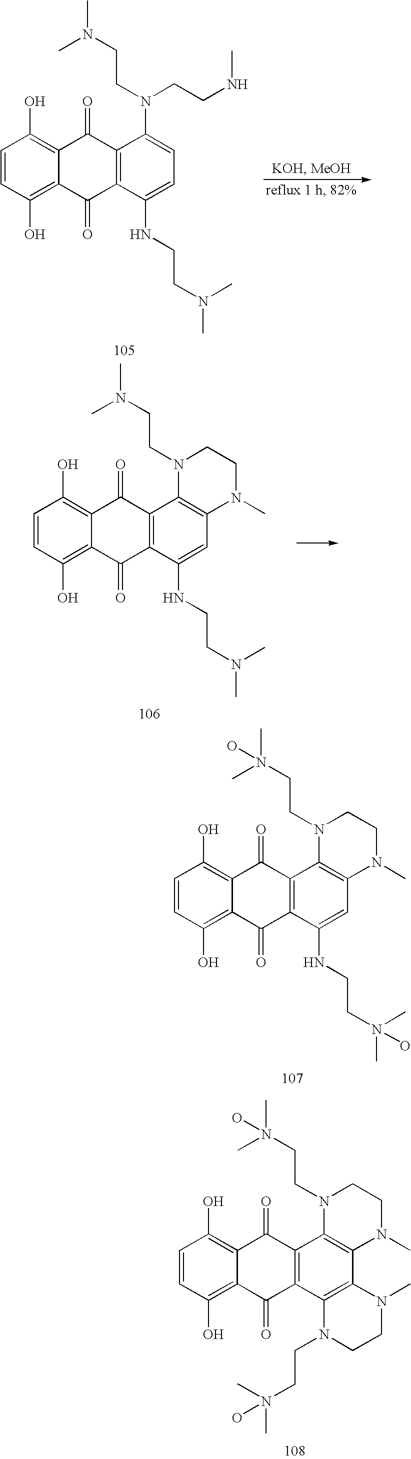

A further variation leading to a more rigid diamine unit than that in AQ4N itself will be obtained by fusing a piperidine ring to the C1-C2 bond as exemplified by AQ4N analog (117) (Scheme 12). Alternatively, a piperidine ring will be fused to both the C1-C2 bond and the C3-C4 bond in AQ4N as exemplified by AQ4N analog (119). Analog (117) is prepared as follows. 1,2,3,4-Tetrahydroquinoline-8-carboxylic acid (Norman et al., J. Med. Chem. 39:4692 (1996)) will be N-benzylated to give (109) and then will be converted into N,N-diethylamide (110). Ortho lithiation (Krapcho et al. Synthetic Commun. 20:2139 (1990)) and then carboxylation will give diacid (111). Fluorination of (111) (or the corresponding anhydride) in the position para to the activating amino group with fluorinating agent (6) (Umemoto et al., Org. Synth. Coll. Vol. 8, 286) followed by heating will give fluoro cyclic anhydride (112). Reaction of (112) with hydroquinone (25) as per Lee and Denny will give anthraquinone (113). Catalytic debenzylation to amine (114) followed by N-alkylation with 1-bromo-2-(N,N-dimethylamino)ethane will give diamine (115). Displacement of the fluorine atom with N,N-dimethylethylenediamine (3) as per Lee and Denny will give tetramine (116). Bis-N-Oxidation of (116) with MCPBA will give AQ4N analog (117).

AQ4N analog (119) will be prepared as follows. 1,2,3,4,7,8,9,10-Octahydro-4,7-phenanthroline (120) (Smith et al., J. Am. Chem. Soc. 74:1096 (1952)) will be bis-N-alkylated with 1-bromo-2-(N,N-dimethylamino)ethane to give tetramine (121). Monobromination of the activated aromatic ring will give bromide (122), which will be lithiated and then carboxylated will give carboxylic acid (123). This compound will be converted into N,N-diethylamide (124) and will be ortho lithiated and then carboxylated to give carboxylic acid (125). Hydrolysis to the diacid followed by heating will give the substituted phthalic anhydride (126). Reaction of (126) with one or the other of the hydroquinones represented by (81) followed by bis-N-oxidation with MCPBA will provide a general route to AQ4N analogs represented by (119).

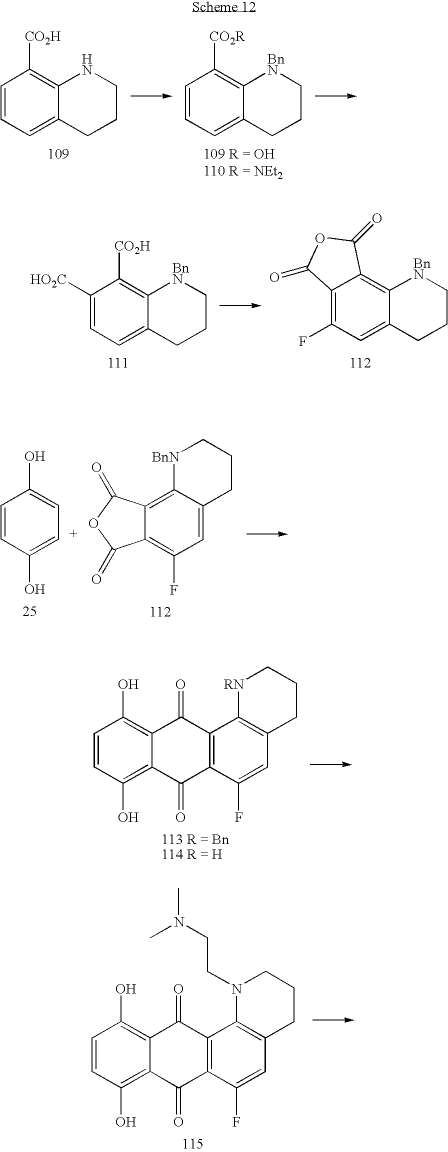

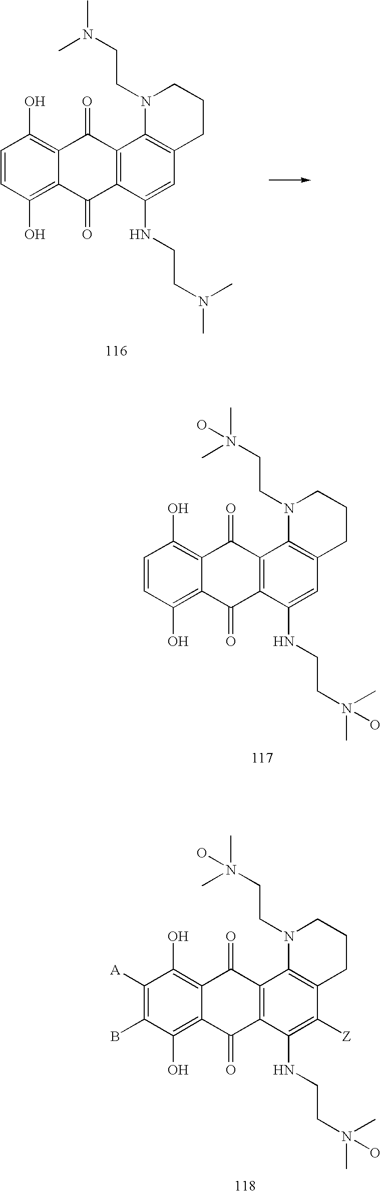

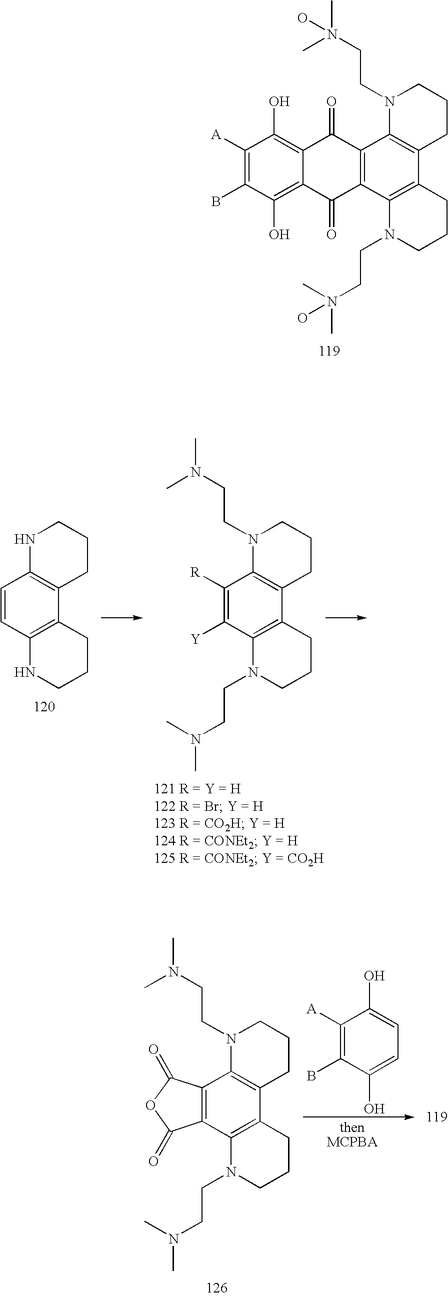

A further variation leading to a more rigid diamine unit than that in AQ4N itself will be obtained by the following routes. Difluoride (16) will be reacted with 1 equiv. of the appropriately N-substituted 2-aminothiophenol (127) under basic conditions (JP 63081164 A2) to give (128) (Scheme 13). Reaction of (128) with a second equiv. of (127) will give (129). MCPBA oxidation then will give the AQ4N analog (130). If instead, (128) will be treated with diamine (3) then one will obtain mixed AQ4N analog (131) after MCPBA oxidation.

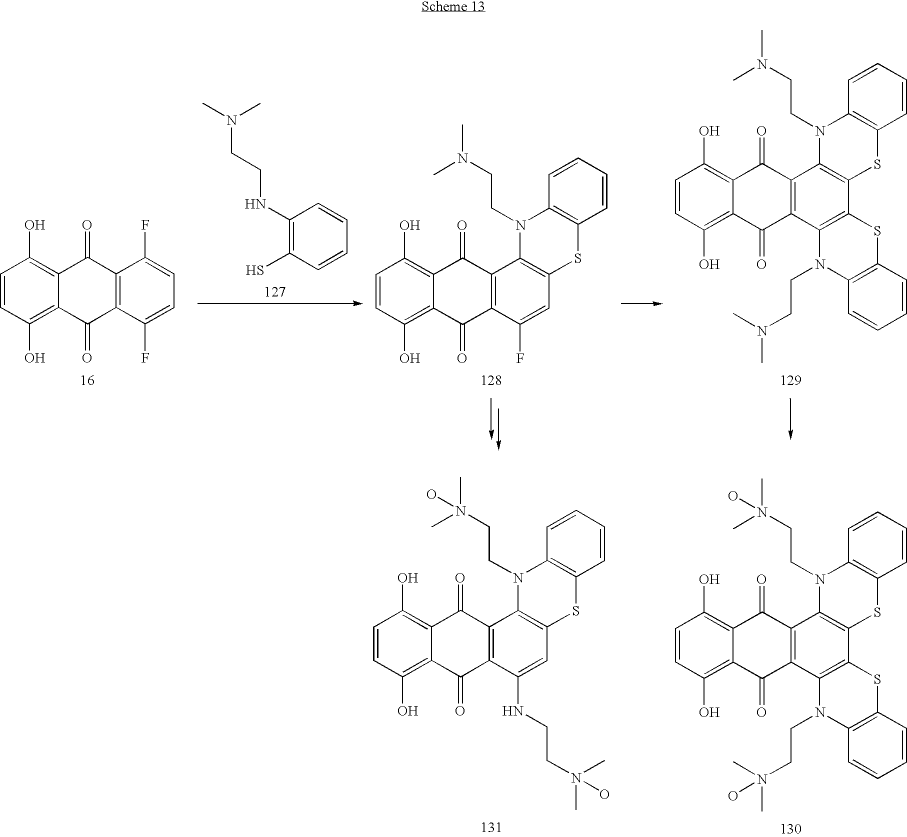

A further variation leading to AQ4N analogs with additional rings is as follows. Replacement of phthalic anhydride (11), for example, with a phthalic anhydride containing an additional fused ring, for example, (132)-(135), will lead to analogs of the above AQ4N molecules containing a fused aryl or heteroaryl ring attached to C2-C3 as shown in Scheme 14.

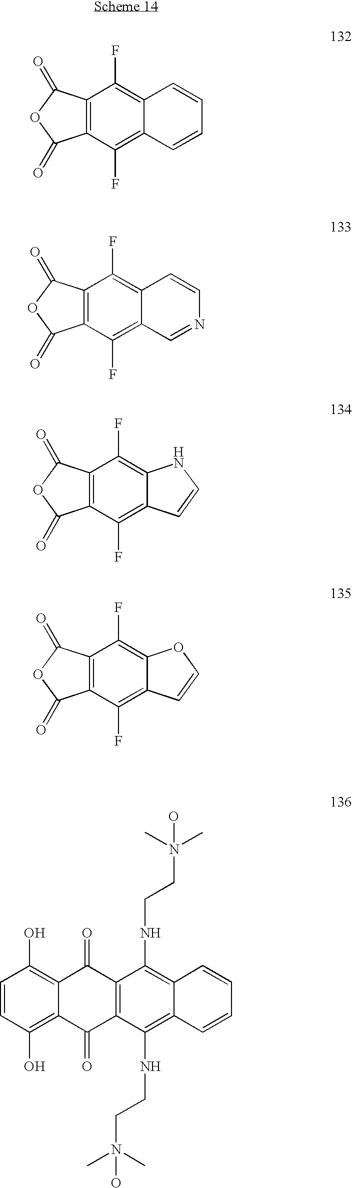

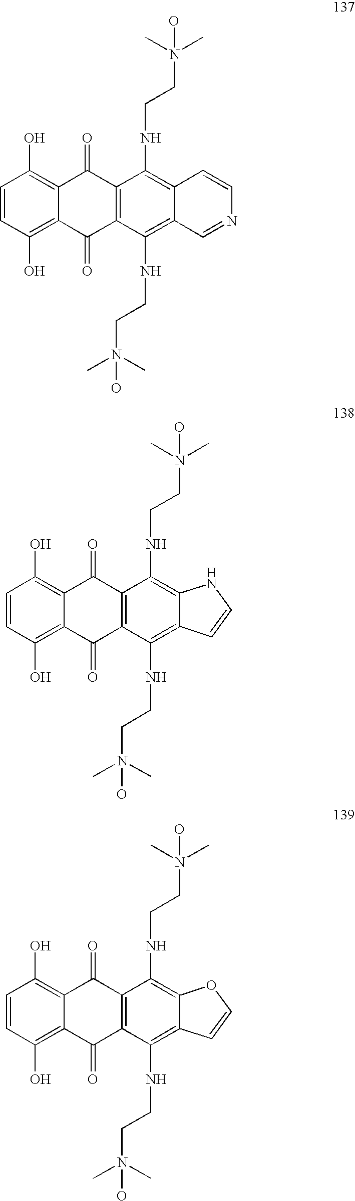

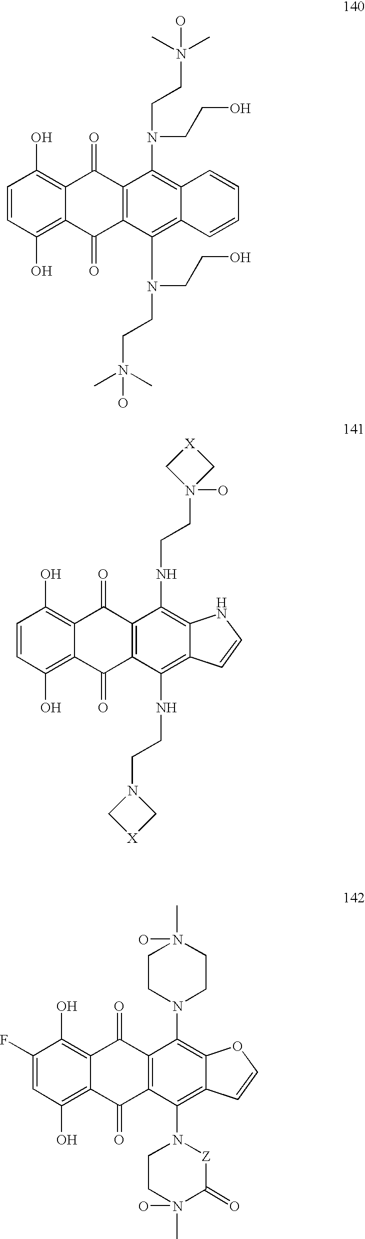

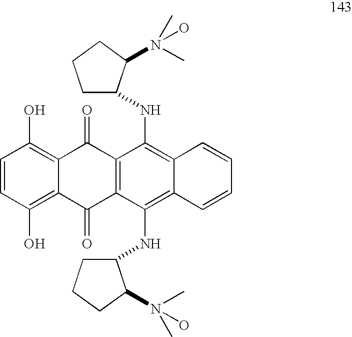

### Scheme 15: Route to Analogs of AQ4N Containing Fluorine in the N-Oxide Region of the Molecule

Replacement of diamine (3) with a fluorine containing diamine such as (144)-(147) in one or the other of the above Schemes will lead to AQ4N analogs which contain fluorine in the N-oxide region of the molecule as shown in Scheme 15. N-Oxides (148) and (149) are examples of AQ4N analogs thus prepared. Fluorine may serve to alter the reduction potential of the molecule.

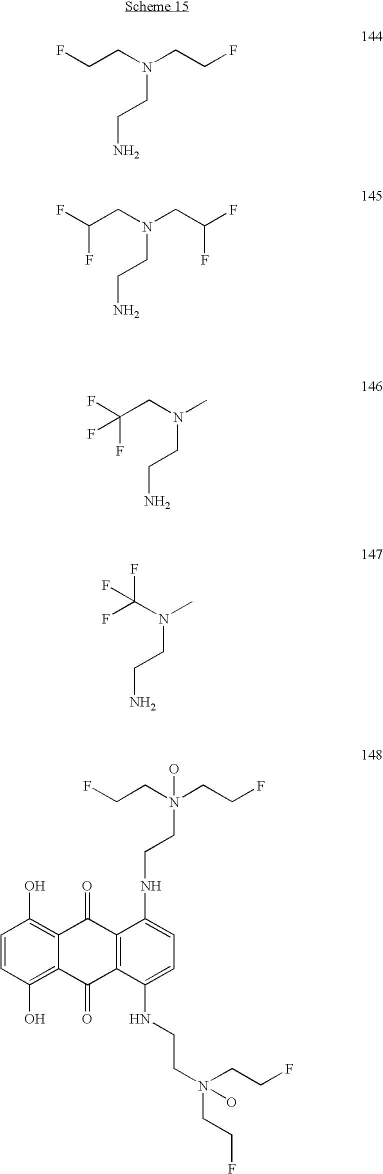

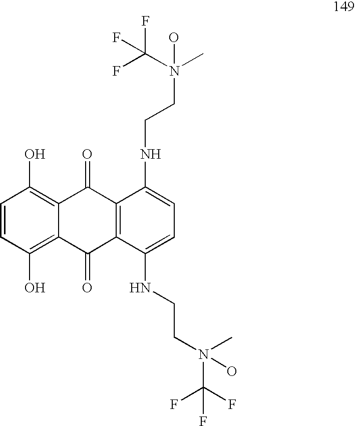

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A compound having Formula I: 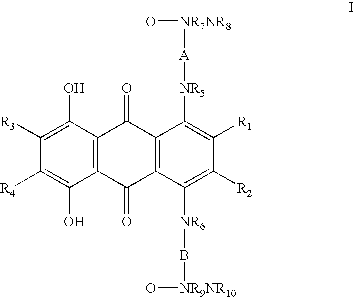
or a pharmaceutically acceptable salt thereof, wherein:
R₁ and R₂ are independently hydrogen, halo, alkyl, carboxyl, carboxylester, carboxylamide, thioalkyl, hydroxy, alkoxy, aryloxy, sulfonyl, sulfonic acid, or polyethylene glycol, or R₁ and R₂ together form an aryl group;
R₃ and R₄ independently are hydrogen or fluorine, or R₃ and R₄ together form an aryl group;
R₅ and R₆ independently are hydrogen, alkyl, or hydroxyalkyl;
or R₁ and R₅ together, and/or R₂ and R₆ together, form a ring;
R₇ and R₉ are independently alkyl, hydroxyalkyl, haloalkyl, or with the neighboring nitrogen form a heterocycle;
R₉ and R₁₀ are independently alkyl, hydroxyalkyl, haloalkyl, or together with the neighboring nitrogen form a heterocycle;
A and B independently are (CH₂)ₙ, cycloalkyl, or aryl, or with the two neighboring nitrogens forms a heterocycle; and
n is 1, 2, 3, or 4;
with the proviso that at least one of R₁, R₂, R₃, R₄, R₅ and R₆ is other than hydrogen; or
at least one of R₇, R₈, R₉, and R₁₀ is haloalkyl;
or R₇ and R₈, and/or R₉ and R₁₀, form a heterocycle with the neighboring nitrogen; or
at least one of A and B is cycloalkyl, heterocyclic or aryl or forms a heterocycle with the two neighboring nitrogens.

2. The compound of claim 1, having Formula II: 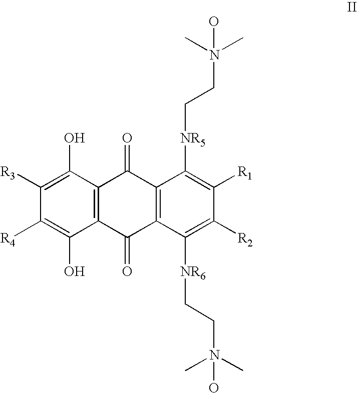
wherein R₁ is hydrogen, halo or optionally substituted alkyl or alkoxy, and R₂, R₃, and R₄ are hydrogen or halo.

3. The compound of claim 2, wherein said compound is selected from:

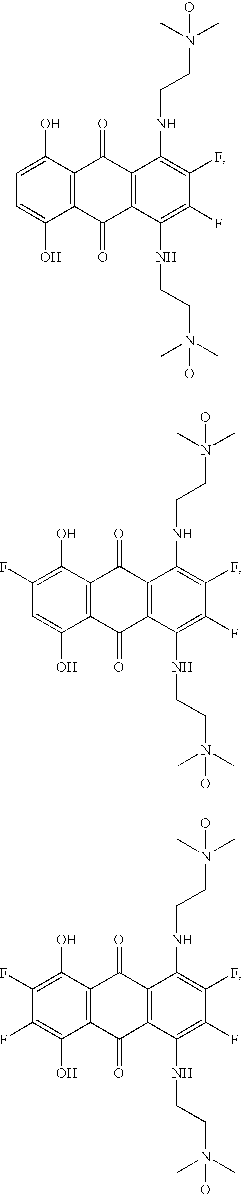

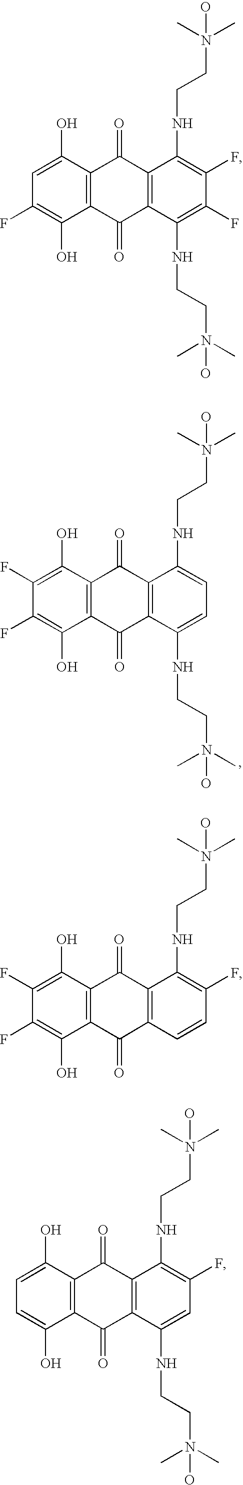

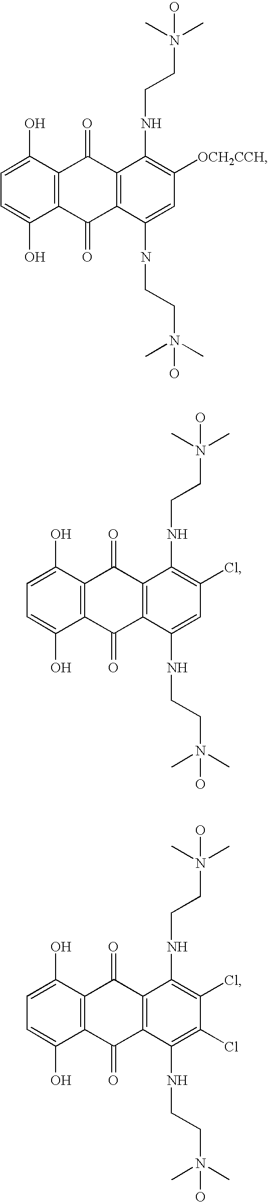

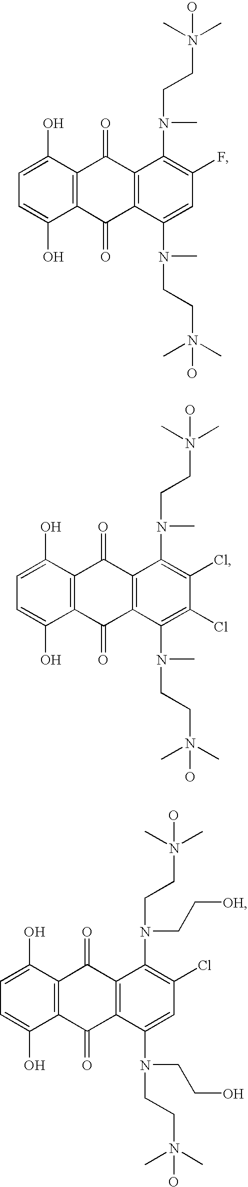

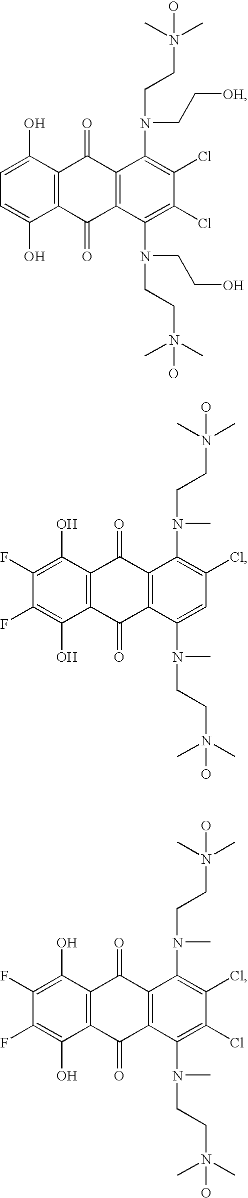

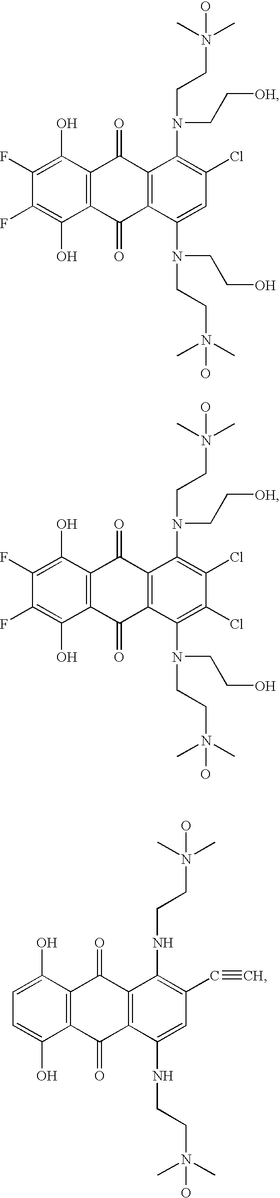

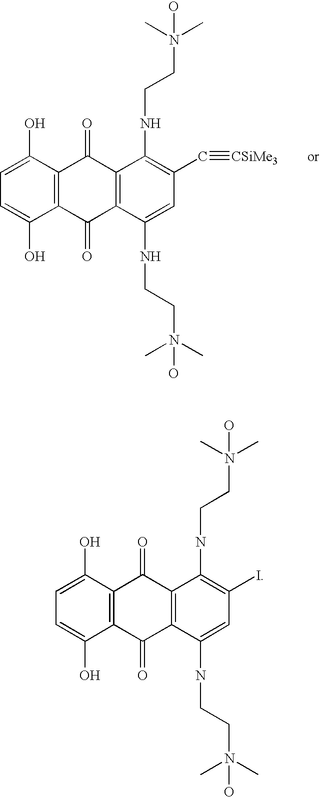

4. The compound of claim 1, having Formula III: 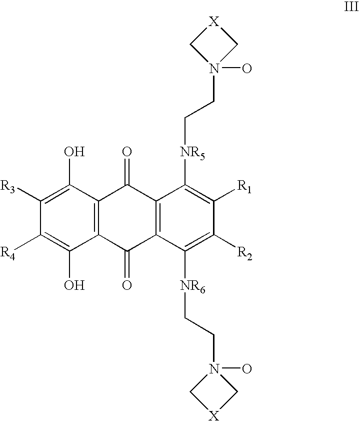
wherein X is (CH₂)ₘ and m is 0 to 5.

5. The compound of claim 4, wherein said compound is selected from:

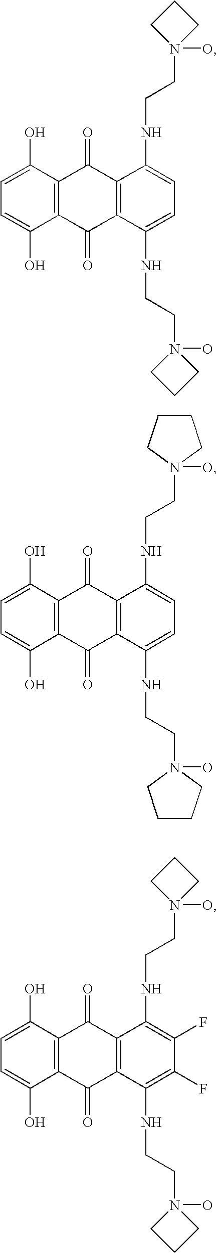

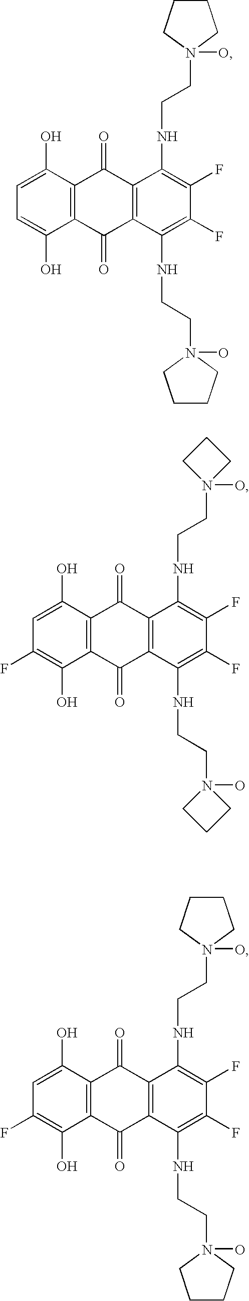

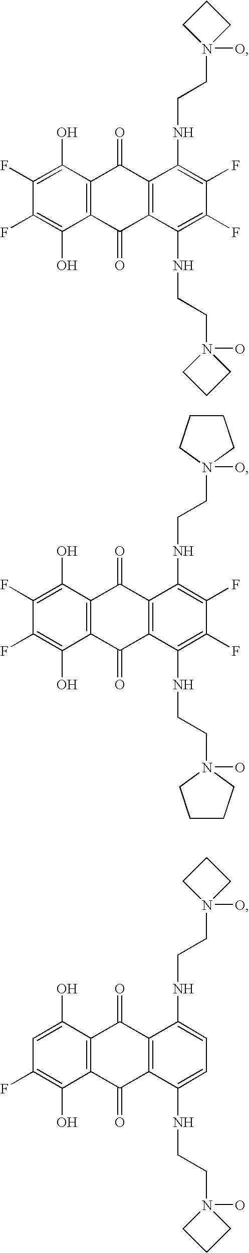

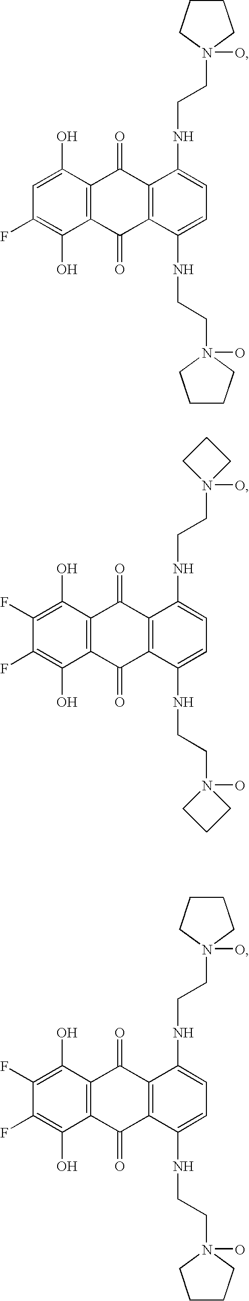

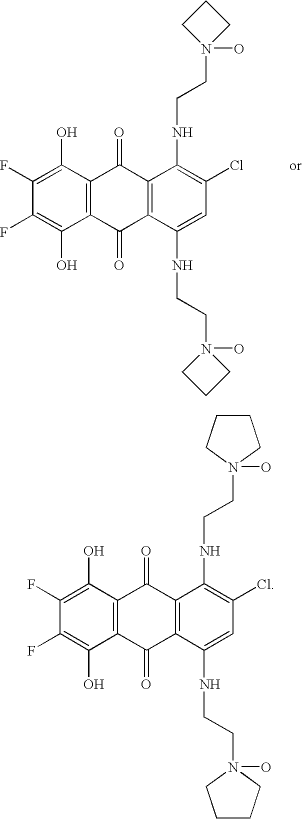

6. The compound of claim 1, having Formula IV: 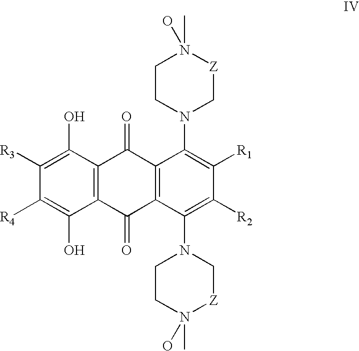
wherein Z is (CH₂)ₘ and m is 0 to 5.

7. The compound of claim 6, wherein said compound is selected from:

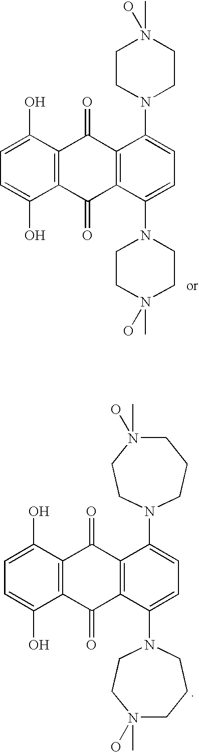

8. The compound of claim 1, having Formula V: 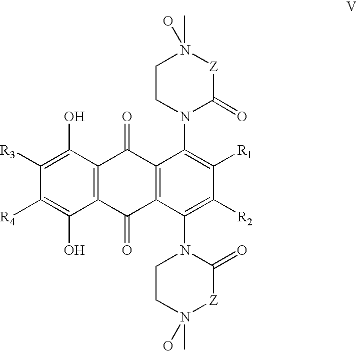
wherein Z is (CH₂)ₘ and m is 0 to 5.

9. The compound of claim 8, wherein said compound is selected from:

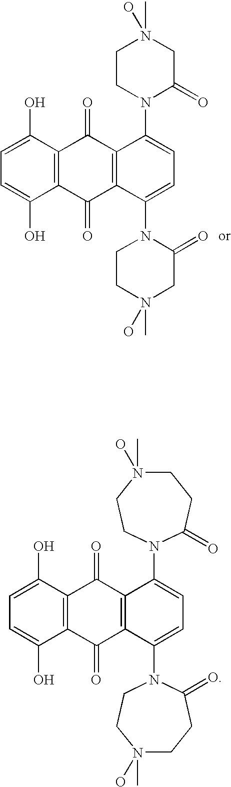

10. The compound of claim 1, having Formula VI: 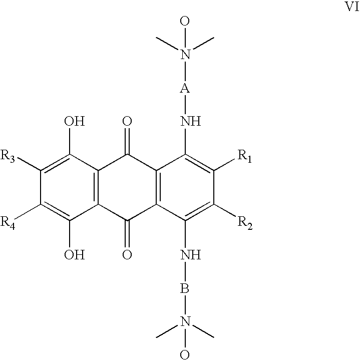
wherein A and B are independently cycloalkyl or heterocyclic groups containing 4-12 ring atoms.

11. The compound of claim 10, wherein said compound is selected from:

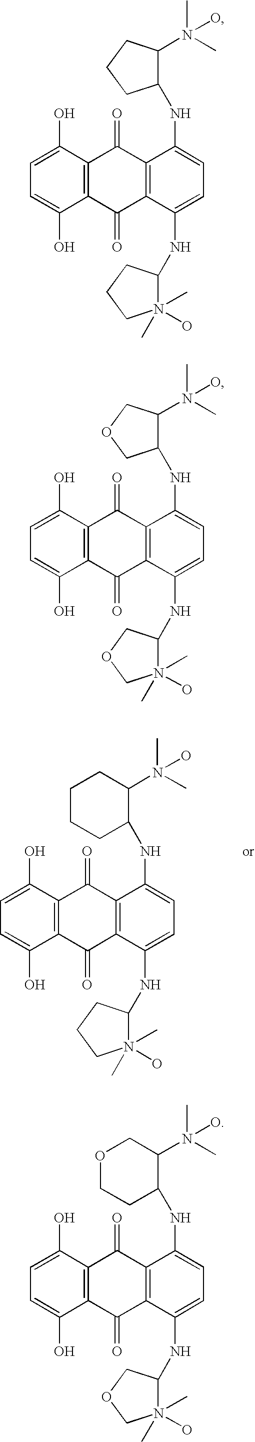

12. The compound of claim 1, having Formula VII: 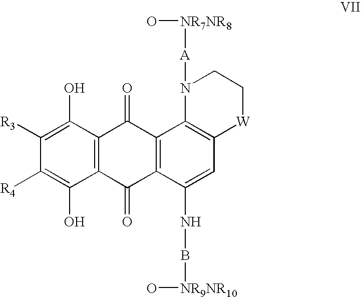
wherein W is CH₂, O, S, or NR₁₁, wherein R₁₁ is hydrogen or alkyl.

13. The compound of claim 12, wherein said compound is selected from:

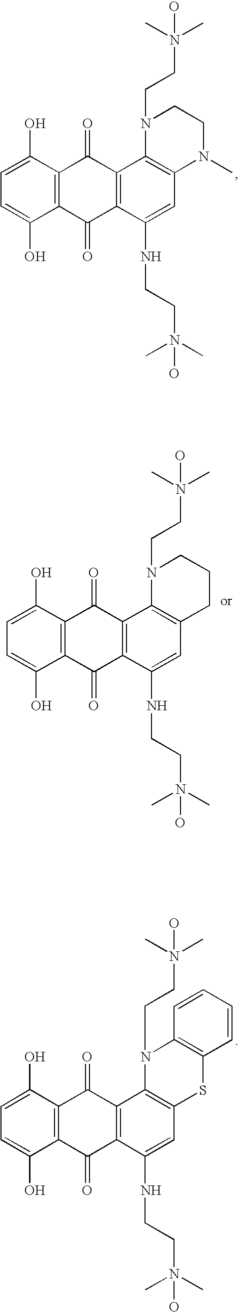

14. The compound of claim 1, having Formula VIII: 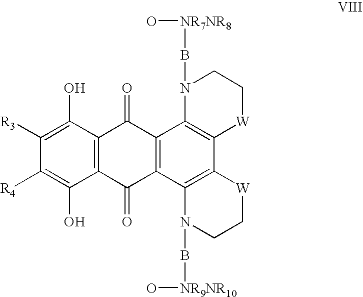
wherein W is CH₂, O, S, or NR₁₁, wherein R₁₁ is hydrogen or alkyl.

15. The compound of claim 14, wherein said compound is selected from:

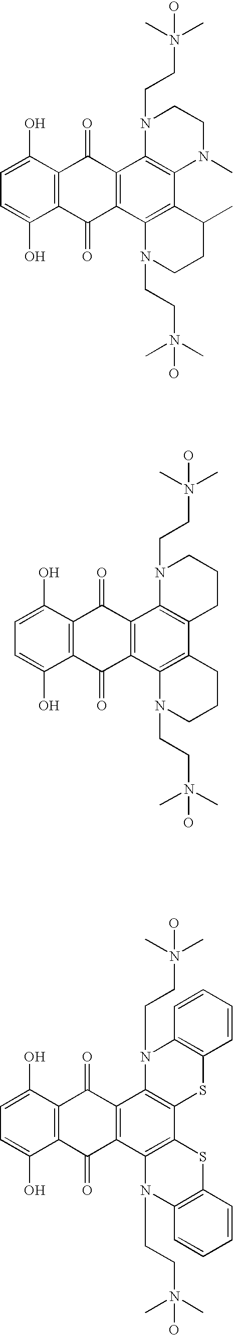

16. The compound of claim 1, having Formula IX: 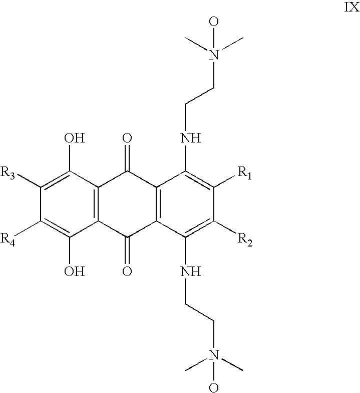
wherein R₁ and R₂ together and/or R₃ and R₄ together form an aryl or heteroaryl group.

17. The compound of claim 16, wherein said compound is selected from:

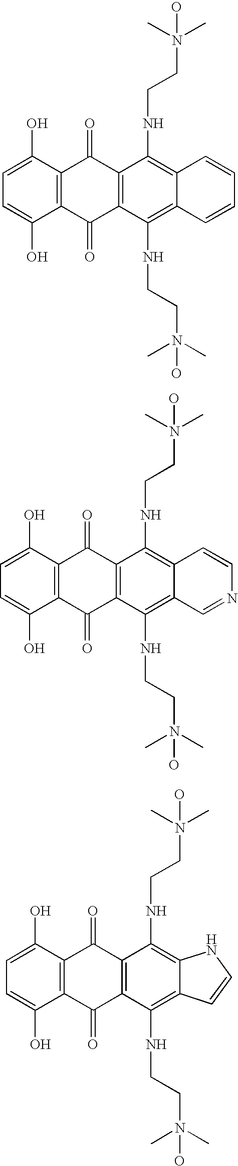

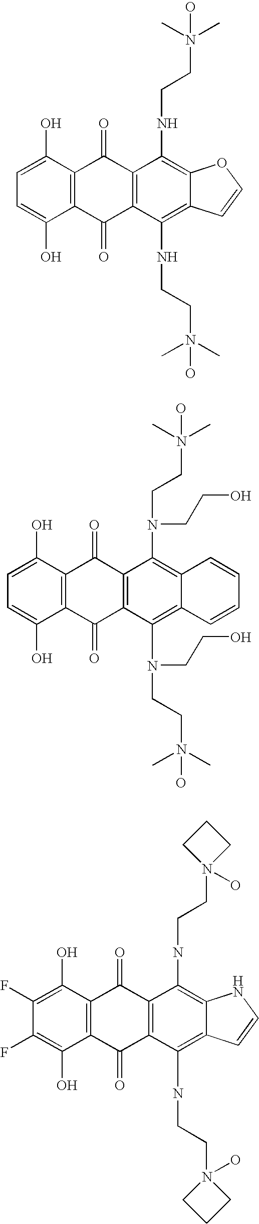

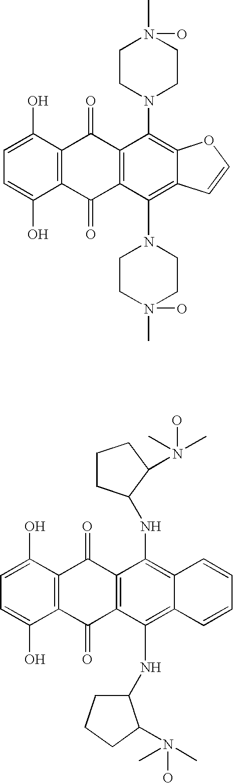

18. The compound of claim 1, having Formula X: 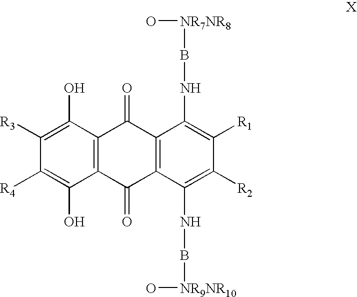
wherein at least one of R₇, R₈, R₉, and R₁₀ is haloalkyl.

19. The compound of claim 18, wherein said compound is selected from:

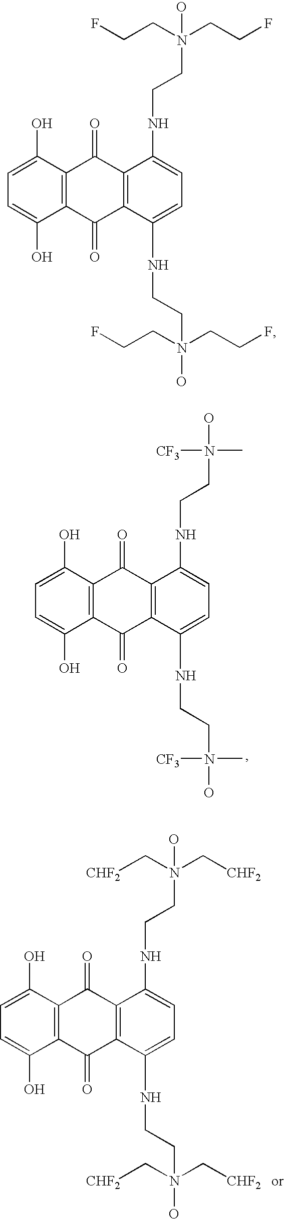

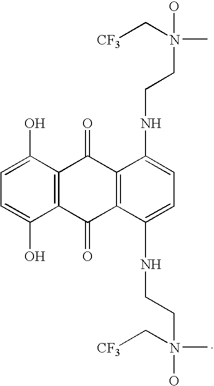

20. A pharmaceutical composition comprising the compound of claim 1.

21. The pharmaceutical composition of claim 20, further comprising one or more other chemotherapeutic or anti-inflammatory agents.

22. The pharmaceutical composition of claim 21, wherein the one or more other chemotherapeutic or anti-inflammatory agents is an active vitamin D compound.

23. The pharmaceutical composition of claim 21, wherein the one or more other chemotherapeutic or anti-inflammatory agents is a chemotherapeutic agent.

24. The pharmaceutical composition of claim 23, wherein the chemotherapeutic agent is selected from the group consisting of abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

25. A method of treating, ameliorating, or preventing a hyperproliferative disorder or cancer comprising administering to a animal in need thereof a therapeutically effective amount of the compound of claim 1.

26. The method of claim 25, wherein the hyperproliferative disorder is cancer.

27. The method of claim 26, wherein the cancer is of the bladder, brain, breast, cervix, colon, endometrium, esophagus, head and neck, kidney, larynx, liver, lung, oral cavity, ovaries, pancreas, prostate, skin, stomach, or testis.

28. The method of claim 26, wherein the cancer is selected from the group consisting of acute and chronic lymphocytic leukemia, acute granulocytic leukemia, adrenal cortex carcinoma, bladder carcinoma, breast carcinoma, cervical carcinoma, cervical hyperplasia, choriocarcinoma, chronic granulocytic leukemia, chronic lymphocytic leukemia, colon carcinoma, endometrial carcinoma, esophageal carcinoma, essential thrombocytosis, genitourinary carcinoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, multiple myeloma, mycosis fungoides, myeloid and lymphocytic leukemia, neuroblastoma, non-Hodgkin's lymphoma, osteogenic sarcoma, ovarian carcinoma, pancreatic carcinoma, polycythemia vera, primary brain carcinoma, primary macroglobulinemia, prostatic carcinoma, renal cell carcinoma, rhabdomyosarcoma, skin cancer, small-cell lung carcinoma, soft-tissue sarcoma, squamous cell carcinoma, stomach carcinoma, testicular carcinoma, thyroid carcinoma, and Wilms' tumor.

29. The method of claim 25, wherein the hyperproliferative disorder is age-related macular degeneration, Crohn's disease, cirrhosis, a chronic inflammatory-related disorder, diabetic retinopathy, granulomatosis, inflammatory bowel disease, psoriasis, rheumatoid arthritis, systemic lupus erythematosus, vascular hyperproliferation secondary to retinal hypoxia, or vasculitis.

30. The method of claim 25, wherein the hyperproliferative disorder is an immunoproliferative disorder.

31. The method of claim 30, wherein the hyperproliferative disorder is immune hyperproliferation associated with organ or tissue transplantation.

32. The method of claim 25, further comprising administering one or more other active agents or treatments to the animal.

33. The method of claim 32, wherein the one or more other active agents or treatments is an active vitamin D compound.

34. The method of claim 32, wherein the one or more other active agents or treatments are independently selected from the group consisting of a chemotherapeutic agent and a radiotherapeutic agent/treatment.

35. The method of claim 34, wherein both one or more chemotherapeutic agents and one or more radiotherapeutic agents/treatments are administered.

36. The method of claim 32, wherein said compound having Formula I is administered prior to the administration of said active agents or treatments.

37. The method of claim 32, wherein said compound having Formula I is administered concurrently with the administration of said active agents or treatments.

38. The method of claim 37, wherein the administration of said compound having Formula I is continued beyond the administration of said active agents or treatments.

39. The method of claim 32, wherein said compound having Formula I is administered after the administration of said active agents or treatments.

40. The method of claim 32, wherein the method is repeated at least once.
